(19) United States
(12) Patent Application Publication
(10) Pub. No.: US 2020/0292554 A1
(43) Pub. Date: Sep. 17, 2020

(54) IMMUNE AND GROWTH-RELATED BIOMARKERS ASSOCIATED WITH PRETERM BIRTH ACROSS SUBTYPES AND PREECLAMPSIA DURING MID-PREGNANCY, AND USES THEREOF

(71) Applicants: The Regents of the University of California, Oakland, CA (US); University of Iowa Research Foundation, Iowa city, IA (US)

(72) Inventors: Laura Jelliffe, San Francisco, CA (US); Kelli Ryckman, Iowa City, IA (US); Jeffrey Murray, Iowa City, IA (US)

(21) Appl. No.: 16/652,400

(22) PCT Filed: Oct. 1, 2018
(86) PCT No.: PCT/US2018/053773

§ 371 Date: Mar. 30, 2020

Related U.S. Application Data
(60) Provisional application No. 62/566,468, filed on Oct. 1, 2017.

(51) Int. Cl. G01N 33/68; A61K 31/57; A61K 9/00; G16H 50/30; G16H 10/40

(52) U.S. Cl. CPC G01N 33/689; A61K 31/57; A61K 9/0036; G01N 2800/60; G16H 10/40; G01N 2800/368; G16H 50/30

(57) ABSTRACT

The disclosure provides for immune- or growth-related biomarkers that are associated with preterm birth across subtypes and preeclampsia, methods of using said biomarkers, including assessing a subject's risk for preterm birth, and prophylactic treatment of the subject based upon the assessment of a greater than average risk for preterm birth using said biomarkers.

### CROSS REFERENCE TO RELATED APPLICATIONS

This application claims priority under 35 U.S.C. § 119 from Provisional Application Ser. No. 62/566,468 filed Oct. 1, 2017, the disclosures of which are incorporated herein by reference.

### STATEMENT OF GOVERNMENT SUPPORT

This invention was made with Government support under Grant Nos. HL101748, R01 HD057192, and R01 HD052953 awarded by the National Institutes of Health. The Government has certain rights in the invention.

### TECHNICAL FIELD

The disclosure provides for immune- or growth-related biomarkers that are associated with preterm birth across subtypes and preeclampsia, methods of using said biomarkers, including assessing a subject's risk for preterm birth, and prophylactic treatment of the subject based upon the assessment of a greater than average risk for preterm birth using said biomarkers.

### BACKGROUND

Worldwide, more than 15 million babies are born preterm (before 37 completed weeks of gestation) each year. Preterm birth (PTB) and its related complications are the leading cause of death in children less than five years of age and contribute to more than 1 million deaths per year. Survivors of PTB are more likely to suffer from both short- and long-term morbidities including blindness, deafness, neurodevelopmental delay, psychiatric disturbance, and diabetes and heart disease in later life.

### SUMMARY

The disclosure provides for immune- or growth-related biomarkers that are associated with preterm birth across subtypes and preeclampsia. The disclosure further provides methods of using said biomarkers in predictive models in order to assess a subject's risk for preterm birth (all subtypes)±preeclampsia. Such an assessment can include the assigning of a risk assessment score that indicates the probability of the subject having preterm birth (all subtypes)±preeclampsia. Moreover, a subject which is deemed to have a greater than average risk for preterm birth (all subtypes)±preeclampsia using the methods disclosed herein, can then be prophylactically treated in attempts to prevent the subject in having a preterm birth.

In particular, the disclosure presents an exemplary study in which 400 women with singleton deliveries in California in 2009-2010 (200 PTB and 200 term) were divided into training and testing samples at a 2:1 ratio. Sixty-three markers were tested in 15-20 serum samples using multiplex technology. Linear discriminate analysis was used to create a discriminate function. Model performance was assessed using area under the receiver operating characteristic curve (AUC). It was found herein that twenty-five serum biomarkers along with maternal age <34 years and poverty status identified >80% of women with PTB±preeclampsia with best performance in women with preterm preeclampsia (AUC=0.889, 95% confidence interval (0.822-0.959) training; 0.883 (0.804-0.963) testing). Accordingly, the immune and growth-related biomarkers of the disclosure reliably identified most women who went on to have a PTB±preeclampsia, especially when the secondary indicators of maternal age and poverty status were considered with the biomarker results.

The disclosure provides a method of generating a risk assessment score for preterm birth (all subtypes)±preeclampsia, for a biological sample obtained from a pregnant female subject, comprising measuring the level of a panel of immune and/or growth-related biomarkers from a biological sample obtained from a pregnant female subject; assigning a risk indicator value or predictor for each of the measured immune and/or growth-related biomarkers; inputting the obtained risk indicator values into a computer implemented predicative multivariate logistic model that is built using a training set and a testing set from a population of pregnant female subjects that comprise subjects that had preterm births and subjects that did not have preterm births; and calculating a risk assessment score for the biological sample obtained from a pregnant female subject using the predictive model, wherein the panel of immune and/or growth-related biomarkers comprises the biomarkers for

Resistin, sFASL, FGF-Basic, and SCF. In one embodiment, the panel of immune and/or growth-related biomarkers further comprises biomarkers for GP130, ENA-78, NGF, PDGFBB, MIG and IL-4. In another or further embodiment, the panel of immune and/or growth-related biomarkers further comprises biomarkers for IL-4R, IL-5, IL-13, IL-17, RAGE, VEGFR3, and RANTES. In another or further embodiment, the panel of immune and/or growth-related biomarkers further comprises biomarkers for PAI1, G-CSF, IL-1R2, IL-17F, IFNB, M-CSF, Eotaxin, and MIP1B. In another or further embodiment, the panel of immune and/or growth-related biomarkers consists essentially of Resistin, sFASL, FGF-Basic, SCF, GP130, ENA-78, NGF, PDGFBB, MIG, IL-4, IL-4R, IL-5, IL-13, IL-17, RAGE, VEGFR3, RANTES, PAI1, G-CSF, IL-1R2, IL-17F, IFNB, M-CSF, Eotaxin, and MIP1B. In another or further embodiment, the panel of immune and/or growth-related biomarkers consists of Resistin, sFASL, FGF-Basic, SCF, GP130, ENA-78, NGF, PDGFBB, MIG, IL-4, IL-4R, IL-5, IL-13, IL-17, RAGE, VEGFR3, RANTES, PAI1, G-CSF, IL-1R2, IL-17F, IFNB, M-CSF, Eotaxin, and MIP1B. In another or further embodiment, the biological sample is a serum sample. In another or further embodiment, the biological sample is a sample obtained from a pregnant female subject that has less than 32 weeks of gestation. In another or further embodiment, the biological sample is a sample obtained from a pregnant female subject that 15 to 20 weeks of gestation. In another or further embodiment, the panel of biomarkers are measured using a quantitative multiplex assay. In another or further embodiment, the quantitative multiplex assay is a quantitative bead-based multiplex immunoassay. In another or further embodiment, the predicative multivariate logistic model is a linear discriminant analysis model. In another or further embodiment, the linear discriminant analysis model uses the coefficients for the biomarkers presented in Table 1 In another or further embodiment, the predictive multivariate logistic model uses the coefficients for the biomarkers presented in Table 1. In another or further embodiment, the method further comprises, assessing the pregnant female subject for any secondary risk factors, including maternal characteristics, medical history, past pregnancy history, obstetrical history, income status, alcohol, tobacco or drug use, diabetes, hypertension, and interpregnancy interval; assigning a risk indicator value for each secondary risk factors; inputting the obtained risk indicator values for the secondary risk factors along with the obtained risk indicator values for the biomarkers into the computer implemented predicative multivariate logistic model; and calculating a risk assessment score for the biological sample obtained from a pregnant female subject using the predictive model. In another or further embodiment, the method uses risk indicator values or predictors for the pregnant female subject being >34 years of age, and/or for the pregnant female subject having a low-income status.

The disclosure provides a method for prophylactically treating a pregnant female subject for preterm birth, comprising determining a risk assessment score from a biological sample obtained from the pregnant female subject using the method(s) as described above; administering a treatment to the pregnant female subject if the risk assessment score for the subject sample indicates that the subject has a high probability for preterm birth, wherein the treatment is selected from progesterone, cervical pessary, cervical cerclage, tocolytic administration, and antibiotic therapy.

The disclosure also provides a kit for assessing preterm birth and preeclampsia risk biomarkers in a sample, wherein the kit comprises a detecting agent(s) for each biomarker in a panel of biomarkers consisting essentially of Resistin, sFASL, FGF-Basic, SCF, GP130, ENA-78, NGF, PDGFBB, MIG, IL-4, IL-4R, IL-5, IL-13, IL-17, RAGE, VEGFR3, RANTES, PAI1, G-CSF, IL-1R2, IL-17F, IFNB, M-CSF, Eotaxin, and MIP1B. In one embodiment, the detecting agents are antibodies. In another or further embodiment, the kit is an ELISA or antibody microarray.

### DESCRIPTION OF DRAWINGS

FIG. 1 presents a flow diagram indicating the sample selection for the model.

FIG. 2 presents the serum markers that were measured in banked 15-20-week serum samples.

FIG. 3 provides the correlations across markers in the final model (training set).

FIG. 4 provides area under the receiver operating characteristic curves (AUCs) for mid-pregnancy immune and growth factor preterm birth±preeclampsia test. Training set AUC (top): 0.803 (95% CI 0.748-0.858); Testing set AUC (bottom): 0.750 (95% CI 0.676-0.825).

FIG. 5 provides a graph of the true and false-positive rates by probability cut-points based on mid-pregnancy immune and growth factor preterm birth±preeclampsia test.

### DETAILED DESCRIPTION

As used herein and in the appended claims, the singular forms “a,” “an,” and “the” include plural referents unless the context clearly dictates otherwise. Thus, for example, reference to “a cytokine” includes a plurality of such cytokines and reference to “the biomarker” includes reference to one or more biomarkers and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the disclosure, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

As used herein, the term “amount” or “level” in reference of an immune- or growth-related biomarker, refers to a quantity of the immune- or growth-related biomarker that is detectable or measurable in a biological sample and/or control.

As used herein, the term “biological sample” includes any sample that is taken from a subject which contains one or more of the immune- or growth-related biomarkers listed in Table 1, Table 3 or Table 4. Suitable samples in the context of the present disclosure include, for example, blood, plasma, serum, amniotic fluid, vaginal excretions, saliva, and urine. In some embodiments, the biological sample is selected from the group consisting of whole blood, plasma, and serum. In a particular embodiment, the biological sample is serum. As will be appreciated by those skilled in the art, a biological sample can include any fraction or component of blood, without limitation, T cells, monocytes, neutrophils, erythrocytes, platelets and microvesicles such as exosomes and exosome-like vesicles.

As used herein, the term “immune- or growth-related biomarker panel,” refers to a collection of two or more immune- or growth-related biomarkers described more fully below. The number of biomarkers useful for an immune- or growth-related biomarker panel is further described herein, and can be based on values or factors, such as values or factors that are grouped based upon p-values for significance that are associated for PTB across subtypes±preeclampsia, or the sharing of a protein motif, e.g., interleukins.

As used herein, the terms “isolated” and “purified,” generally describes a composition of matter that has been removed from its native environment (e.g., the natural environment if it is naturally occurring), and thus is altered by the hand of man from its natural state. An isolated protein or nucleic acid is distinct from the way it exists in nature. Thus, for example, purified cDNA obtained by RT-PCR, or antibody captured polypeptides or purified polypeptides are contemplated herein. Such nucleic acids, polypeptide, antibodies etc. can be detectably labeled for optical measurements, radioisotope measurements etc. Such detectable labels do not “naturally occur” on such polypeptide, nucleic acid, antibodies and the like.

As used herein, “low income-status” or “poverty” refers to a person that earns a gross monthly income that is less than 138% of the federal poverty level for a specific household size. Typically, a person who has “low income-status” or is “poor” for this disclosure receives some form of government assistance (e.g., “Medi-Cal” payments) and/or receives some form of federal assistance (e.g., Supplemental Nutrition Assistance Program, Temporary Assistance for Needy Families, refugee benefits, etc.).

As used herein, the term “mass spectrometer” refers to a device able to volatilize/ionize analytes to form gas-phase ions and determine their absolute or relative molecular masses. Suitable methods of volatilization/ionization are matrix-assisted laser desorption ionization (MALDI), electrospray, laser/light, thermal, electrical, atomized/sprayed and the like, or combinations thereof. Suitable forms of mass spectrometry include, but are not limited to, ion trap instruments, quadrupole instruments, electrostatic and magnetic sector instruments, time of flight instruments, time of flight tandem mass spectrometer (TOF MS/MS), Fourier-transform mass spectrometers, Orbitraps and hybrid instruments composed of various combinations of these types of mass analyzers. These instruments can, in turn, be interfaced with a variety of other instruments that fractionate the samples (for example, liquid chromatography or solid-phase adsorption techniques based on chemical, or biological properties) and that ionize the samples for introduction into the mass spectrometer, including matrix-assisted laser desorption (MALDI), electrospray, or nanospray ionization (ESI) or combinations thereof.

The terms “patient”, “subject” and “individual” are used interchangeably herein, and refer to an animal, particularly a human. This includes human and non-human animals. The term “non-human animals” and “non-human mammals” are used interchangeably herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. In a particular embodiment, the subject is a female subject. In a further embodiment, the subject is a pregnant female subject. In yet a further embodiment, the subject is a pregnant female human subject. In a particular embodiment, the subject is a pregnant human female subject having a gestational period of less than 32 weeks. In a further embodiment, the subject is a pregnant human female subject having a gestational period between 32 to 36 weeks.

As used herein, “preeclampsia” refers a pregnancy complication characterized by high blood pressure and signs of damage to another organ system, most often the liver and kidneys. Preeclampsia usually begins after 20 weeks of pregnancy in women whose blood pressure had been normal.

As used herein, “PPTB” refers to a suite of pregnancy complications that includes PTB (birth occurring at fewer than 37 weeks gestational age) and preeclampsia.

As used herein, “PTB” includes both spontaneous PTB (preterm premature rupture of membranes and/or preterm labor), and induced PTB (medical induction or cesarean section due to medical indication).

Preterm birth refers to delivery or birth at a gestational age less than 37 completed weeks. Other commonly used subcategories of preterm birth have been established and delineate moderately preterm (birth at 33 to 36 weeks of gestation), very preterm (birth at <33 weeks of gestation), and extremely preterm (birth at ≤28 weeks of gestation). Gestational age is a proxy for the extent of fetal development and the fetus's readiness for birth. Gestational age has typically been defined as the length of time from the date of the last normal menses to the date of birth. However, obstetric measures and ultrasound estimates also can aid in determining gestational age. Preterm births have generally been classified into two separate subgroups. One, spontaneous preterm births are those occurring subsequent to spontaneous onset of preterm labor or preterm premature rupture of membranes regardless of subsequent labor augmentation or cesarean delivery. Two, indicated preterm births are those occurring following induction or cesarean section for one or more conditions that the woman's caregiver determines to threaten the health or life of the mother and/or fetus.

As used herein, a “risk indicator” refers to a factor that is predictive for PTB across subtypes±preeclampsia in a pregnant subject. Risk indicators may comprise various immune- or growth-related biomarkers described herein, wherein the presence or abundance of the immune- or growth-related biomarker is indicative of an increased or decreased risk for PTB across subtypes±preeclampsia. Risk indicators may also include maternal characteristics, such as health history, health status, age; drug, tobacco or alcohol abuse; unfavorable demographics, e.g., low income status, etc. A more complete listing of risk indicators is further provided herein.

Worldwide, more than 15 million babies are born preterm (before 37 completed weeks of gestation) each year. Preterm birth (PTB) and its related complications are the leading cause of death for children less than five years of age and contribute to more than one million deaths per year. Survivors of PTB are more likely to suffer from both short and long-term morbidities including blindness, deafness, neurodevelopmental delay, psychiatric disturbance, diabetes, and heart disease in later life. While all neonates born preterm are at risk for short and long-term morbidity and mortality, those with early PTB (gestational age (GA), <32 weeks) are at the highest risk. Spontaneous PTB resulting from premature labor or preterm premature rupture of membranes (PPROM) is the most common clinical presentation of PTB. This type of PTB occurs in approximately two in three pregnancies with preterm delivery in the United States and in other high-income countries and in more than three in four pregnancies delivering preterm in low-and middle-income countries. Other PTBs generally result from cesarean delivery or induction due to provider determination of maternal or fetal indication.

Despite increased clinical, research, and policy focus, rates of PTB are increasing worldwide, including in the United States. After several years of decline, the rate of PTB in the United States increased in 2015, which continued into 2016.

The continuing burden of PTB despite increased focus suggests the need for a different approach to addressing PTB from a research, clinical, and policy perspective. While historically, prevention efforts have focused mostly on women with a previous PTB or short cervix, or have focused on extending gestational duration in women with early signs of labor, there is a growing push for management based on a woman's specific personal risk profile. In 2016, the Society for Maternal Fetal Medicine (SMFM) released its first PTB Toolkit which outlines recommended management of women based on a number of risk factors for PTB (e.g., bacteriuria, smoking, obesity, pregestational diabetes, and chronic hypertension).

Consideration of a clinical shift to address the risk of PTB has also recently begun to be considered for women testing as “high-risk” based on mid-pregnancy biomarkers. In general, the principle behind such tests is that they might allow for the identification of at-risk pregnant women that may otherwise go unidentified. A test that identifies pregnant women who are more likely to deliver early and spontaneously and excludes those likely to deliver at term may also hold potential from a patient education and clinical surveillance perspective—particularly with respect to recognition of early signs of labor including cervical shortening, PPROM, or contractions. Moreover, women that do not exhibit other traditional risks (e.g., previous PTB, short cervix) likely would benefit from existing therapies (e.g., progesterone, cervical pessary, cervical cerclage, tocolytic administration, and antibiotic therapy). These efforts are closely aligned with those focused on early identification of pregnancies at increased risk for preeclampsia (ending in preterm and term birth) given the established efficacy of aspirin administration 16-weeks for reducing recurrence.

Recent years have seen progress in the development of PTB prediction test with three tests in or moving into the market. Two existing tests measure proteins and microparticles identified by using mass spectrometry, while another test uses Q-PCR to measure circulating cell-free plasma RNAs in order to identify women at increased risk for spontaneous PTB. Currently these tests focus mostly on spontaneous PTB (PTB related to preterm premature rupture of membranes (PPROM) or premature labor) and generally do not address provider initiated PTB (PTB resulting from cesarean section or induction due to fetal or maternal indication). Efforts focused on molecular and other prediction testing for preeclampsia are also well underway but also rarely address overlap with efforts aimed at predicting PTB.

While existing prediction tests for spontaneous PTB (and for preeclampsia not associated with PTB) demonstrate the promise of using mid-pregnancy biomarkers for prediction purposes, these tests, however, are not generally applicable to all forms of PTBs. Given the breadth of data demonstrating common pathophysiological underpinning across spontaneous and provider-initiated subtypes of PTB including among those that include or do not include preeclampsia, it appears possible that a predictive test could be developed that covers a wider range of PTB phenotypes. For example, all PTB subtypes including those that include or do not include preeclampsia, have been shown to have strong links to markers of immune function (e.g., cytokines and chemokines) and to angiogenic growth factors (e.g., vascular endothelial growth factor (VEGF)). Moreover, the existing tests rely on advanced -omic platforms, there also appears to be an opportunity to develop a test that relies on lower cost technology (e.g., multiplex) that is more widely available and as such, may maximize the potential for translation both in the United States and in other developed and developing settings.

It was postulated that a comprehensive test for PTB across multiple subtypes, including±preeclampsia, could be developed based upon mid-pregnancy growth factors and immune-related factors, along with maternal demographics and obstetric factors. The disclosure demonstrates that when considered in combination, maternal characteristics and serum immune and growth-related markers can be used at 15-20 weeks of gestation to identify women that have an increased risk for PTB occurring±preeclampsia. The resulting linear discriminate analysis (LDA) PTB±preeclampsia model was able to consistently identify more than three and four women going on to deliver preterm across training and testing subsets with the best performance for preterm preeclampsia where AUCs were consistently at or above 88%.

The methods disclosed herein were able to reliably specify a woman's magnitude of risk for PTB±preeclampsia with higher probabilities associated with lower term false-positive rates. For example, while >60% of women going on to have a PTB±preeclampsia had a 15-20 week LDA-derived probability score≥0.5 so did >28% of pregnancies going on to have a term delivery. While the detection rate was far lower at higher probability cut-points, so was the rate of false positives in term pregnancies. For instance at a LDA-derived probability score ≥0.8, detection rates for PTB were consistently above 25% and detection rates for PTB with preeclampsia were consistently above 35% with false-positive rates in pregnancies going to term that were consistently below 5%.

Heretofore, the disclosure provides prediction for PTB across subtypes±preeclampsia. Given that the AUCs from the studies described herein equaled or exceeded those of investigations focused on, for example, spontaneous PTB or preeclampsia it appears that such an approach may offer similar predictive capacity and broader applicability over other serum testing approaches.

For example, using circulating proteins, investigators were able to identify women with a spontaneous PTB <37 weeks with an observed AUC of 0.75, while other investigators were able to identify women with a spontaneous PTB<37 weeks with an observed AUC of 0.76 using cell-free plasma RNAs (compared with an AUC of 0.81 (rounded) for spontaneous PTB in the training set and 0.84 (rounded) in the testing set in the present study). The results presented herein, with respect to prediction of preterm preeclampsia, also appear to meet or exceed other serum tests for preterm preeclampsia. For example, investigators have reported an AUC of 0.95 for preeclampsia before 32 weeks and an AUC of 0.87 for any preeclampsia before 37 weeks using 11 to 13 week placental growth factor (PLGF) and pregnancy-associated plasma protein A (PAPP-A). It was observed that there was an AUC for preterm preeclampsia of 0.95 (rounded) in the training set and 0.88 (rounded) in the testing set for preeclampsia <32 weeks and we observed an AUC for all preterm preeclampsia (<37 weeks) of 0.89 in the training sample and 0.88 in the testing sample. The methods disclosed herein perform as well or better for all births <37 weeks than other serum tests known in the art that are specific to spontaneous PTB and preeclampsia.

Accordingly, the methods disclosed herein represent an improvement over other methods taught in the art given that the methods disclosed herein focus on the commonalities across PTB subtypes and relies on widely available multiplex technology that allows multiple markers to be measured in a single test, further benefits may be realized if the methods of the disclosure were focused within subtypes. Accordingly, the methods disclosed herein can be further improved by the inclusion of, for example, a second-tier -omics-based test that addresses other protein-based or metabolic factors. A second-tier test that included ultrasound measures might also increase detection rates for preterm preeclampsia. Such an approach might allow for broad testing for baseline all PTB±preeclampsia risk and second-tier testing that is specifically aimed at early PTBs and preterm preeclampsia with a focus on term false-positive reduction.

Provided herein are methods comprising immune and growth-related biomarker panels that have been shown herein to have significant association with a subject's risk for pregnancy complications, which includes PTB risk across subtypes (including spontaneous PTB and induced PTB) and the development of preeclampsia. The methods disclosed herein may further comprise secondary risk indicators, including maternal age >34 years and low-income status, which have also been shown herein to be predictive for pregnancy complications. The method of the disclosure is capable of assessing the cumulative risk for all subtypes of PTB and the pregnancy complication of eclampsia, which is heretofore was not available or known in the art. The immune and growth-related biomarker panels and methods of the disclosure can be readily implemented with a single assay and provides early assessment of a subject's pregnancy complication risk in a convenient and quick manner, allowing for expedited treatment of the subject to prevent the occurrence of the pregnancy complications.

In particular embodiments, the disclosure provides for methods comprising immune and growth-related biomarker panels that can be used for predicting the risk of PPTB in a subject, in other words, the risk that the subject will experience PTB and/or preeclampsia. The methods disclosed herein, in part, are based upon the derivation of predictive relationships between certain indicators and PPTB risk found in the studies presented herein. Notably, the disclosure provides methods for the assessment of PPTB risk across numerous underlying factors, providing a comprehensive and integrated means to assess PPTB risk in the general population using a novel combinations of risk indicators.

Accordingly, the disclosure is based, in part, on the discovery that certain immune- and/or growth-related biomarkers in a biological sample obtained from a pregnant female are differentially expressed in pregnant females that have an increased risk for PTB across subtypes±preeclampsia relative to matched controls. It was further found herein, that the predictability of a subject's risk for PTB across subtypes±preeclampsia using the methods disclosed herein, can be further improved when the assessment of the immune- and/or growth-related biomarker panels described herein is used in combination with other non-biomarker risk factors, including, but not limited to, the subject's age (e.g., >34 years of age); use of alcohol or tobacco; preexisting or existing condition (e.g., diabetes, hypertension, etc.); use of drugs, whether illicit or otherwise; self or family history of PTB; interpregnancy interval (IPI) <12 months; obesity (body mass index (BMI) 30 m/kg²); and income-status.

The disclosure provides biomarker panels, methods and kits for determining the probability for PTB across subtypes±preeclampsia in a pregnant female. One major advantage of the biomarker panels, methods and kits disclosed herein is that the risk of a pregnant subject in developing PTB across subtypes±preeclampsia can be assessed early on in pregnancy, so that appropriate monitoring and clinical management to prevent PTB can be initiated in a timely and preventive fashion. Thus, the biomarker panels, methods and kits disclosed herein is of particular benefit to females that lack other risk factors (e.g., self or family history of PTB, short cervix, preexisting conditions, drug and alcohol abuse, etc.) for preterm birth and who would not otherwise be identified and treated.

By way of example, the disclosure includes methods for generating a result useful in determining probability for PTB across subtypes±preeclampsia in a pregnant female by obtaining a dataset associated with a sample, where the dataset at least includes quantitative data about immune- and/or growth-related biomarkers and panels of immune- and/or growth-related biomarkers that have been identified herein as predictive of PTB across subtypes±preeclampsia, and inputting the dataset into an analytic process that uses the dataset to generate a result useful in determining probability for PTB across subtypes±preeclampsia in a pregnant female.

In addition to the specific biomarkers identified in this disclosure, for example, the polynucleotide and polypeptide sequence of which are publicly available in electronic databases, e.g., GenBank, Euroepan Nucleotide Archive, DNA Data Bank of Japan, UniProt, Swiss-Prot, TrEMBL, Protein Information Resource, Protein Data Bank, Ensembl, and InterPro. The disclosure also contemplates use of biomarker variants that are at least 90% or at least 95% or at least 97% identical to the exemplified sequences provided in the publicly available databases, and that are now known or later discovered and that have utility for the methods disclosed herein. These variants may represent polymorphisms, splice variants, mutations, and the like. In this regard, the disclosure presents multiple art-known proteins in the context of the biomarker panels and methods disclosed herein. However, those skilled in the art will appreciate that accession numbers and journal articles can easily be identified that can provide additional characteristics of the disclosed immune- and/or growth-related biomarkers and that the exemplified references are in no way limiting with regard to the disclosed biomarkers. As described herein, various techniques and reagents find use in the methods disclosed herein. Suitable samples in the context of the present disclosure include, for example, blood, plasma, serum, amniotic fluid, vaginal excretions, saliva, and urine. In some embodiments, the biological sample is selected from the group consisting of whole blood, plasma, and serum. In a particular embodiment, the biological sample is serum. As described herein, immune- and/or growth-related biomarkers can be detected through a variety of assays and techniques known in the art. As further described herein, such assays include, without limitation, mass spectrometry (MS)-based assays, antibody-based assays as well as assays that combine aspects of the two.

Immune- and/or growth-related biomarkers associated with the probability for PTB across subtypes±preeclampsia in a pregnant female include, but are not limited to, one or more of the isolated immune- and/or growth-biomarkers listed in Table 1, Table 3 or Table 4. In addition to the specific immune- and/or growth-related biomarkers, the disclosure further includes immune- and/or growth-related biomarker variants that are about 90%, about 95%, or about 97% identical to the exemplified sequences. Variants, as used herein, include polymorphisms, splice variants, mutations, and the like.

Additional secondary risk indicators for PTB across subtypes±preeclampsia can be selected from one or more non-biomarker risk indicators, including but not limited to, maternal characteristics, medical history, preexisting conditions (e.g., diabetes, hypertension, etc.), past pregnancy history, obstetrical history, and income status. Such additional risk indicators can include, but are not limited to, a self or family history of previous low birth weight or preterm delivery; multiple 2nd trimester spontaneous abortions; prior first trimester induced abortion; history of infertility; nulliparity; placental abnormalities; cervical and uterine anomalies; gestational bleeding; intrauterine growth restriction; in utero diethylstilbestrol exposure; multiple gestations; infant sex; low pre-pregnancy weight/low body mass index; diabetes; hypertension; urogenital infections; obesity (body mass index (BMI) 30 m/kg²); interpregnancy interval (IPI) <12 months; low-income status; maternal age; employment-related physical activity; occupational exposures and environment exposures; inadequate prenatal care; cigarette smoking; use of over the counter medications and/or prescribed drugs; use of illicit drugs; alcohol consumption; caffeine intake; dietary intake; sexual activity during late pregnancy; and leisure-time physical activities. Additional risk indicia useful for as markers can be identified using learning algorithms known in the art, such as linear discriminant analysis, support vector machine classification, recursive feature elimination, prediction analysis of microarray, logistic regression, CART, FlexTree, LART, random forest, MART, and/or survival analysis regression, which are known to those of skill in the art and are further described herein.

Provided herein are panels of isolated immune- and/or growth-related biomarkers comprising N of the biomarkers selected from the group listed in Table 1, Table 3 or Table 4. In the disclosed panels of biomarkers N can be a number selected from the group consisting of 2 to 25. In the disclosed methods, the number of biomarkers that are detected and whose levels are determined, can be 1, or more than 1, such as 2, 3, 4, 5, 6, 7, 8, 9, 10, 11, 12, 13, 14, 15, 16, 17, 18, 19, 20, 21, 22, 23, 24, or 25, or a range that includes, or is between, any two of foregoing values (e.g., 2-5, 2-10, 2-15, 2-20, 2-25, 3-5, 3-10, 3-15, 3-20, 3-25, 4-5, 4-10, 4-15, 4-20, 4-25, 5-10, 5-15, 5-20, 5-25, 6-10, 6-15, 6-20, 6-25, 7-10, 7-15, 7-20, 7-25, 8-10, 8-15, 8-20, 8-25, 9-10, 9-15, 9-20, 9-25, 10-15, 10-20, or 10-25). It should be appreciated that the foregoing provides non-limiting examples of possible ranges, and it is fully contemplated herein that additional ranges are included in this disclosure besides the ones specially recited above.

In further embodiments, the disclosed methods further comprise the assessment of non-biomarker risk indicators, as indicated above. Accordingly, the number of non-biomarker risk indicators that are assessed and whose levels are determined, can be 1, or more than 1, such as 2, 3, 4, 5, 6, 7, 8, 9, 10, 15, 20, 25, 30 or a range that includes, or is between, any two of foregoing values (e.g., 2 to 10). For example, the methods of the disclosure can further comprise assessing non-biomarker risk indicators, such as low-income status, drug use, preexisting diabetes, preexisting hypertension, reported smoking, obesity (body mass index (BMI) 30 m/kg²), interpregnancy interval (IPI) <12 months, parity, and previous PTB.

While certain of the immune- and/or growth-related biomarkers listed in Table 1, Table 3 or Table 4, are useful alone for determining the probability for PTB across subtypes±preeclampsia in a pregnant female, methods are also described herein for the grouping of multiple subsets of the biomarkers that are each useful as one or more panels of biomarkers. Such panels of biomarkers can be based upon sharing a common protein motif, as is presented in Table 3, e.g., interleukins, chemokine ligands, etc. Alternatively, the panels of biomarkers can be based upon grouping biomarkers based upon a p-cutoff value for association for PTB across subtypes±preeclampsia (e.g., see Table 4). For example, a method disclosed herein can comprise a first panel that comprises immune- and/or growth-related biomarkers that have p-value of 0.01 for significance of association for PTB across subtypes±preeclampsia, such as Resistin, sFASL, FGF-Basic, and SCF; a second panel of immune- and/or growth-related biomarkers that have p-value from 0.02 to 0.05 for significance of association for PTB across subtypes±preeclampsia, such as GP130, ENA-78, NGF, PDGFBB, MIG and IL-4; a third panel of immune- and/or growth-related biomarkers that have p-value from 0.06 to 0.10 for significance of association for PTB across subtypes±preeclampsia, such as IL-4R, IL-5, IL-13, IL-17, RAGE, VEGFR3, and RANTES; and a fourth panel of immune- and/or growth-related biomarkers that have p-value from 0.10 to 1 for significance of association for PTB across subtypes±preeclampsia, such as PAI1, G-CSF, IL-1R2, IL-17F, IFNB, M-CSF, and Eotaxin. The disclosure also contemplates that combinations of panels (see above) can be used such the first panel and second panel; first panel and third panel; first panel, second panel and third panel; first panel and fourth panel; first panel, second panel and fourth panel; first panel, third panel and fourth panel; and first panel, second panel, third panel and fourth panel.

The disclosure also provides a method of determining probability for PTB across subtypes±preeclampsia in a pregnant female, the method comprising measuring the amounts of immune or growth-related biomarkers selected from Table 1, Table 3, or Table 4 from a subject's biological sample. In some embodiments, the disclosed methods for determining the probability of PTB across subtypes±preeclampsia encompass detecting and/or quantifying one or more immune or growth-related biomarkers using detection agents or equipment, such as mass spectrometry, a capture agent or a combination thereof.

In some embodiments, the disclosed methods of determining probability for PTB across subtypes±preeclampsia in a pregnant female encompass an initial step of providing an immune or growth-related biomarker panel comprising N of the biomarkers listed in Table 1, Table 3, or Table 4. In additional embodiments, the disclosed methods of determining probability for PTB across subtypes±preeclampsia in a pregnant female encompass an initial step of providing a biological sample from the pregnant female.

In some embodiments, the disclosed methods of determining the probability for PTB across subtypes±preeclampsia in a pregnant female encompass communicating the probability to a health care provider. In additional embodiments, the communication informs a subsequent treatment decision for the pregnant female. In some embodiments, the method of determining probability for PTB across subtypes±preeclampsia in a pregnant female encompasses the additional feature of expressing the probability as a risk score. The term “risk score” refers to a score that can be assigned based on comparing the amount of one or more immune- or growth-related biomarkers in a biological sample obtained from a pregnant female subject to a standard or reference score that represents an average amount of the one or more biomarkers calculated from biological samples obtained from a random pool of pregnant females or a pool of pregnant females that reached full-term. Because the level of an immune- or growth-related biomarker may not be static throughout pregnancy, a standard or reference score can be obtained for the gestational time point that corresponds to that of the pregnant female at the time the sample was taken. The standard or reference score can be predetermined and built into a predictor model such that the comparison is indirect rather than actually performed every time the probability is determined for a subject. A risk score can be a standard (e.g., a number) or a threshold (e.g., a line on a graph). The value of the risk score correlates to the deviation, upwards or downwards, from the average amount of the one or more immune- or growth-related biomarkers calculated from biological samples obtained from a random pool of pregnant females. In certain embodiments, if a risk score is greater than a standard or reference risk score, the subject has an increased likelihood for PTB across subtypes±preeclampsia. In some embodiments, the magnitude of a pregnant female's risk score, or the amount by which it exceeds a reference risk score, can be indicative of or correlated to that pregnant female's level of risk for PTB across subtypes±preeclampsia. In one embodiment, the measurement includes measuring a marker and determining its level and comparing the level to a control, wherein if the test sample level varies (depending upon the marker) up or down by greater than 2% (e.g., 5%, 10%, 15%, 20%, 25%, 30%, 35%, 40%, 45%, 50%, 55%, 60%, 65%, 70%, 75%, 80%, 85%, 90%, 95%, or any value between any of the foregoing), a “risk” is identified.

In some embodiments, the pregnant female subject was less than 37 weeks of gestation time at the time the biological sample was obtained. In other embodiments, the pregnant female subject was at 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, or a range that includes or is between any two of the foregoing time points, of gestation time at the time the sample was obtained. In a further embodiment, the pregnant female subject was from 32 to 36 weeks of gestation time at the time the biological sample was collected. In further embodiments, the pregnant female subject was less than 32 weeks of gestation time at the time the biological sample was obtained.

In some embodiments, calculating the probability for PTB across subtypes±preeclampsia in a pregnant female is based on the quantified amount of each of N biomarkers selected from the immune- or growth-related biomarkers listed in Table 1, Table 3, or Table 4. Any existing, available or conventional separation, detection and quantification methods can be used herein to measure the presence or absence (e.g., readout being present vs. absent; or detectable amount vs. undetectable amount) and/or quantity (e.g., readout being an absolute or relative quantity, such as, for example, absolute or relative concentration) of immune- or growth-related biomarkers, and/or fragments thereof and optionally of the one or more other biomarkers or fragments thereof in samples. In some embodiments, detection and/or quantification of one or more immune- or growth-related biomarkers comprises an assay that utilizes a capture agent. In further embodiments, the capture agent is an antibody, antibody fragment, nucleic acid-based or protein binding reagent, small molecule or variant thereof. In additional embodiments, the assay is an enzyme immunoassay (EIA), enzyme-linked immunosorbent assay (ELISA), and radioimmunoassay (RIA). In some embodiments, detection and/or quantification of one or more immune- or growth-related biomarkers further comprises mass spectrometry (MS). In yet further embodiments, the mass spectrometry is co-immunoprecipitation-mass spectrometry (co-IP MS), where coimmunoprecipitation, a technique suitable for the isolation of whole protein complexes, is followed by mass spectrometric analysis.

In a particular embodiment, the immune- or growth-related biomarkers can be quantified by mass spectrometric (MS) techniques. Generally, any mass spectrometric (MS) technique that can provide precise information on the mass of peptides, and also on fragmentation and/or (partial) amino acid sequence of selected peptides (e.g., in tandem mass spectrometry, MS/MS; or in post source decay, TOF MS), can be used in the methods disclosed herein. Suitable peptide MS and MS/MS techniques and systems are known (see, e.g., Methods in Molecular Biology, vol. 146: “Mass Spectrometry of Proteins and Peptides”, by Chapman, ed., Humana Press 2000; Biemann 1990. Methods Enzymol 193: 455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more immune or growth-related biomarkers disclosed herein. Such quantitative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS). Other methods useful in this context include isotope-coded affinity tag (ICAT) followed by chromatography and MS/MS.

Mass spectrometry assays, instruments and systems suitable for biomarker peptide analysis can include, without limitation, matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)ₙ (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)ₙ; atmospheric pressure photoionization mass spectrometry (APPI-MS); APPI-MS/MS; and APPI-(MS)ₙ. Peptide ion fragmentation in tandem MS (MS/MS) arrangements can be achieved using manners established in the art, such as, e.g., collision induced dissociation (CID). As described herein, detection and quantification of immune or growth-related biomarkers disclosed herein by mass spectrometry can involve multiple reaction monitoring (MRM), such as described among others by Kuhn et al. Proteomics 4: 1175-86 (2004). Scheduled multiple-reaction-monitoring (Scheduled MRM) mode acquisition during LC-MS/MS analysis enhances the sensitivity and accuracy of peptide quantitation. Anderson and Hunter, Molecular and Cellular Proteomics 5(4):573 (2006). As described herein, mass spectrometry-based assays can be advantageously combined with upstream peptide or protein separation or fractionation methods, such as for example with the chromatographic and other methods described herein below.

A person skilled in the art will appreciate that a number of methods can be used to determine the amount of a biomarker, including mass spectrometry approaches, such as MS/MS, LC-MS/MS, multiple reaction monitoring (MRM) or SRM and product-ion monitoring (PIM) and also including antibody-based methods such as immunoassays such as Western blots, enzyme-linked immunosorbent assay (ELISA), immunoprecipitation, immunohistochemistry, immunofluorescence, radioimmunoassay, dot blotting, and FACS. Accordingly, in some embodiments, determining the level of the at least one immune- or growth-related biomarker comprises using an immunoassay and/or mass spectrometric method. In additional embodiments, the mass spectrometric methods are selected from MS, MS/MS, LC-MS/MS, SRM, PIM, and other such methods that are known in the art. In other embodiments, LC-MS/MS further comprises 1D LC-MS/MS, 2D LC-MS/MS or 3D LC-MS/MS. Immunoassay techniques and protocols are generally known to those skilled in the art (Price and Newman, Principles and Practice of Immunoassay, 2nd Edition, Grove's Dictionaries, 1997; and Gosling, Immunoassays: A Practical Approach, Oxford University Press, 2000.) A variety of immunoassay techniques, including competitive and non-competitive immunoassays, can be used (Self et al., Curr. Opin. Biotechnol., 7:60-65 (1996).

In further embodiments, the immunoassay is selected from Western blot, ELISA, immunoprecipitation, immunohistochemistry, immunofluorescence, radioimmunoassay (RIA), dot blotting, and FACS. In certain embodiments, the immunoassay is an ELISA. In yet a further embodiment, the ELISA is direct ELISA (enzyme-linked immunosorbent assay), indirect ELISA, sandwich ELISA, competitive ELISA, multiplex ELISA, ELISPOT technologies, and other similar techniques known in the art. Principles of these immunoassay methods are known in the art, for example John R. Crowther, The ELISA Guidebook, 1st ed., Humana Press 2000, ISBN 0896037282. Typically, ELISAs are performed with antibodies but they can be performed with any capture agents that bind specifically to one or more biomarkers of the disclosure and that can be detected. Multiplex ELISA allows simultaneous detection of two or more analytes within a single compartment (e.g., microplate well) usually at a plurality of array addresses (Nielsen and Geierstanger 2004. J Immunol Methods 290: 107-20 (2004) and Ling et al. 2007. Expert Rev Mol Diagn 7: 87-98 (2007)).

In some embodiments, Radioimmunoassay (RIA) can be used to detect one or more immune or growth-related biomarkers in the methods disclosed herein. Radioimmunoassay) is a competition-based assay that is known in the art and involves mixing known quantities of radioactively-labelled (e.g., ¹²⁵I or ¹³¹I-labelled) target analyte with antibody specific for the analyte, then adding non-labelled analyte from a sample and measuring the amount of labelled analyte that is displaced (see, e.g., An Introduction to Radioimmunoassay and Related Techniques, by Chard T, ed., Elsevier Science 1995, ISBN 0444821198 for guidance).

A detectable label can be used in the assays described herein for direct or indirect detection of the one or more immune or growth-related biomarkers in the methods disclosed herein. A wide variety of detectable labels can be used, with the choice of label depending on the sensitivity required, ease of conjugation with the antibody, stability requirements, and available instrumentation and disposal provisions. Those skilled in the art are familiar with selection of a suitable detectable label based on the assay detection of the biomarkers in the methods of the disclosure. Suitable detectable labels include, but are not limited to, fluorescent dyes (e.g., fluorescein, fluorescein isothiocyanate (FITC), Oregon Green™, rhodamine, Texas red, tetrarhodamine isothiocyanate (TRITC), Cy3, Cy5, etc.), fluorescent markers (e.g., green fluorescent protein (GFP), phycoerythrin, etc.), enzymes (e.g., luciferase, horseradish peroxidase, alkaline phosphatase, etc.), nanoparticles, biotin, digoxigenin, metals, and the like.

A chemiluminescence assay using a chemiluminescent antibody can be used for sensitive, non-radioactive detection of protein levels. An antibody labeled with fluorochrome also can be suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), beta-galactosidase, urease, and the like. Detection systems using suitable substrates for horseradish-peroxidase, alkaline phosphatase, β-galactosidase are well known in the art.

A signal from the direct or indirect label can be analyzed, for example, using a spectrophotometer to detect color from a chromogenic substrate; a radiation counter to detect radiation such as a gamma counter for detection of ¹²⁵1 (including film measurements followed by density detection); or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, assays used to practice the disclosure can be automated or performed robotically, and the signal from multiple samples can be detected simultaneously. In one embodiment, density, fluorometery etc. measurements are converted to a digital value for comparison.

As described above, chromatography can also be used in practicing the methods disclosed herein. Chromatography encompasses methods for separating chemical substances and generally involves a process in which a mixture of analytes is carried by a moving stream of liquid or gas (“mobile phase”) and separated into components as a result of differential distribution of the analytes as they flow around or over a stationary liquid or solid phase (“stationary phase”), between the mobile phase and said stationary phase. The stationary phase can be usually a finely divided solid, a sheet of filter material, or a thin film of a liquid on the surface of a solid, or the like. Chromatography is well understood by those skilled in the art as a technique applicable for the separation of chemical compounds of biological origin, such as, e.g., amino acids, proteins, fragments of proteins or peptides, etc.

Chromatography can be columnar (i.e., wherein the stationary phase is deposited or packed in a column), liquid chromatography, or by high-performance liquid chromatography (HPLC). Particulars of chromatography are well known in the art (Bidlingmeyer, Practical HPLC Methodology and Applications, John Wiley & Sons Inc., 1993). Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilized metal affinity chromatography, and the like. Chromatography, including single-, two- or more-dimensional chromatography, can be used as a peptide fractionation method in conjunction with a further peptide analysis method, such as for example, with a downstream mass spectrometry analysis as described elsewhere in this specification.

Further peptide or polypeptide separation, identification or quantification methods can be used, optionally in conjunction with any of the above described analysis methods, for measuring immune- or growth-related biomarkers of the disclosure. Such methods include, without limitation, chemical extraction partitioning, isoelectric focusing (IEF) including capillary isoelectric focusing (LIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In the context of the disclosure, the term “capture agent” refers to a compound that can specifically bind to a target, in particular an immune or growth-related biomarker. The term includes antibodies, antibody fragments, nucleic acid-based protein binding reagents (e.g. aptamers, Slow Off-rate Modified Aptamers (SOMAmer™)), protein-capture agents, natural ligands (i.e. a hormone for its receptor or vice versa), small molecules or variants thereof.

Capture agents can be configured to specifically bind to a target, in particular an immune or growth-related biomarker. Capture agents can include but are not limited to organic molecules, such as polypeptides, polynucleotides and other non-polymeric molecules that are identifiable to a skilled person. In the embodiments disclosed herein, capture agents include any agent that can be used to detect, purify, isolate, or enrich a target, in particular an immune or growth-related biomarker. Any art-known affinity capture technologies can be used to selectively isolate and enrich/concentrate biomarkers that are components of complex mixtures of biological media for use in the disclosed methods.

Antibody capture agents that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986). Antibody capture agents can be any immunoglobulin or derivative thereof, whether natural or wholly or partially synthetically produced. All derivatives thereof which maintain specific binding ability are also included in the term. Antibody capture agents have a binding domain that is homologous or largely homologous to an immunoglobulin binding domain and can be derived from natural sources, or partly or wholly synthetically produced. Antibody capture agents can be monoclonal or polyclonal antibodies. In some embodiments, an antibody is a single chain antibody. Those of ordinary skill in the art will appreciate that antibodies can be provided in any of a variety of forms including, for example, humanized, partially humanized, chimeric, chimeric humanized, etc. Antibody capture agents can be antibody fragments including, but not limited to, Fab, Fab′, F(ab′)2, scFv, Fv, dsFv diabody, and Fd fragments. An antibody capture agent can be produced by any means. For example, an antibody capture agent can be enzymatically or chemically produced by fragmentation of an intact antibody and/or it can be recombinantly produced from a gene encoding the partial antibody sequence. An antibody capture agent can comprise a single chain antibody fragment. Alternatively or additionally, antibody capture agent can comprise multiple chains which are linked together, for example, by disulfide linkages; and, any functional fragments obtained from such molecules, wherein such fragments retain specific-binding properties of the parent antibody molecule. Because of their smaller size as functional components of the whole molecule, antibody fragments can offer advantages over intact antibodies for use in certain immunochemical techniques and experimental applications.

It would understood by those skilled in the art that the immune- or growth-related biomarkers disclosed herein can be modified prior to analysis to improve their resolution or to determine their identity. For example, the immune- or growth-related biomarkers can be subject to proteolytic digestion before analysis. Any protease can be used. Proteases, such as trypsin, that are likely to cleave the biomarkers into a discrete number of fragments are particularly useful. The fragments that result from digestion function as a fingerprint for the immune- or growth-related biomarkers, thereby enabling their detection indirectly. This is particularly useful where there are immune- or growth-related biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the immune- or growth-related biomarkers can be modified by the attachment of a tag of particular molecular weight that specifically binds to the immune- or growth-related biomarkers, further distinguishing them. Optionally, after detecting such modified biomarkers, the identity of the immune- or growth-related biomarkers can be further determined by matching the physical and chemical characteristics of the modified biomarkers in a protein database (e.g., SwissProt).

The immune- or growth-related biomarkers identified herein for assessing a subject's risk for PTB across subtypes±preeclampsia in the subject's sample can be captured on a substrate for detection. Traditional substrates include antibody-coated 96-well plates or nitrocellulose membranes that are subsequently probed for the presence of the proteins. Alternatively, protein-binding molecules attached to microspheres, microparticles, microbeads, beads, or other particles can be used for capture and detection of immune- or growth-related biomarkers disclosed herein. The protein-binding molecules can be antibodies, peptides, peptoids, aptamers, small molecule ligands or other protein-binding capture agents attached to the surface of particles. Each protein-binding molecule can include unique detectable label that is coded such that it can be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes. In a particular embodiment, it has been found that the multiple immune or growth-related biomarkers can be advantageously measured or quantified by using a quantitative multiplex assay, for example a direct assay, an indirect assay, a sandwich assay, or a competitive assay, as known in the art, for example, an ELISA assay, wherein the assay elements enable the detection of multiple immune- or growth-related biomarkers as described herein. In one embodiment, the multiplex assay is a bead assay. In another embodiment, the multiplex assay is a Luminex XMAP™ or like assay.

In another embodiments, biochips can be used for capture and detection of the biomarkers of the disclosure. Many protein biochips are known in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture agent bound there. The capture agent can be a biological molecule, such as a polypeptide or a nucleic acid, which captures other biomarkers in a specific manner. Alternatively, the capture agent can be a chromatographic material, such as an anion exchange material or a hydrophilic material. Examples of protein biochips are well known in the art.

Measuring mRNA in a biological sample can be used as a surrogate for detection of the level of the corresponding protein biomarker in a biological sample. Thus, any of the biomarkers or biomarker panels described herein can also be detected by detecting the appropriate RNA. Levels of mRNA can be measured by reverse transcription quantitative polymerase chain reaction (RT-PCR followed with qPCR). RT-PCR is used to create a cDNA from the mRNA. The cDNA can be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

Some embodiments disclosed herein relate to diagnostic and prognostic methods of determining the probability for PTB across subtypes±preeclampsia in a pregnant female subject. The detection of the level of expression of one or more immune or growth-related biomarkers disclosed herein and/or the determination of a ratio of the immune or growth-related biomarkers of the disclosure can be used to determine the probability for PTB across subtypes±preeclampsia in a pregnant female subject. Such detection methods can be used, for example, for early diagnosis of the condition, to determine whether a subject is predisposed to preterm birth, to monitor the progress of preterm birth or the progress of treatment protocols, to assess the severity of preterm birth, to forecast the outcome of preterm birth and/or prospects of recovery or birth at full term, or to aid in the determination of a suitable treatment for preterm birth.

The quantitation of one or more immune or growth-related biomarkers disclosed herein in a biological sample can be determined, without limitation, by the methods described above as well as any other method known in the art. The quantitative data thus obtained is then subjected to an analytic classification process. In such a process, the raw data is manipulated according to an algorithm, where the algorithm has been pre-defined by a training set of data, for example as described in the examples provided herein. An algorithm can utilize the training set of data provided herein, or can utilize the guidelines provided herein to generate an algorithm with a different set of data. In one embodiment, a training set provides a fingerprint-type pattern (e.g., a pattern of values and ranges indicative or normal or risk associated subjects).

In some embodiments, methods disclosed herein that are used to determine the probability for PTB across subtypes±preeclampsia in a pregnant female subject encompasses the use of a predictive model. In further embodiments, methods disclosed herein that are used to determine the probability for PTB across subtypes±preeclampsia in a pregnant female subject encompasses comparing measured immune or growth-related biomarkers with a reference measurement (or pattern of measurements) for said immune or growth-related biomarkers. As those skilled in the art can appreciate, such comparison can be a direct comparison to the reference measurement or an indirect comparison where the reference measurement has been incorporated into the predictive model. In further embodiments, analyzing the measurements of immune or growth-related biomarkers to determine the probability for PTB across subtypes±preeclampsia in a pregnant female subject encompasses one or more of a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, partial least squares-discriminate analysis, multiple linear regression analysis, multivariate non-linear regression, backwards stepwise regression, threshold-based methods, tree-based methods, Pearson's correlation coefficient, Support Vector Machine, generalized additive models, supervised and unsupervised learning models, cluster analysis, or other predictive model known in the art. In particular embodiments, the analysis comprises a linear discriminant analysis model. In further embodiments, the linear discriminant analysis model utilizes the coefficients presented in Table 1.

An analytic classification process can use any one of a variety of statistical analytic methods to manipulate the quantitative data and provide for classification of the sample. Examples of useful methods include a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, partial least squares-discriminate analysis, multiple linear regression analysis, multivariate non-linear regression, backwards stepwise regression, threshold-based methods, tree-based methods, Pearson's correlation coefficient, Support Vector Machine, generalized additive models, supervised and unsupervised learning models, cluster analysis, or other predictive model known in the art.

Classification can be made according to predictive modeling methods that set a threshold for determining the probability that a sample belongs to a given class. The probability preferably is at least 50%, or at least 60%, or at least 70%, or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUROC (area under the ROC curve) or accuracy, of a particular value, or range of values. Area under the curve measures are useful for comparing the accuracy of a classifier across the complete data range. Classifiers with a greater AUC have a greater capacity to classify unknowns correctly between two groups of interest. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC of at least about 0.7, at least about 0.75, at least about 0.8, at least about 0.85, at least about 0.9, or higher. For example, it was observed herein that there was an AUC for preterm preeclampsia of 0.95 (rounded) in the training set and 0.88 (rounded) in the testing set for preeclampsia <32 weeks and we observed an AUC for all preterm preeclampsia (<37 weeks) of 0.89 in the training sample and 0.88 in the testing sample.

The predictive calculations of the model (as well as model generation steps described in the previous section) may be carried out by any suitable digital computer. Suitable digital computers may include portable devices, laptop and desktop computers, cloud computing systems, etc., using any standard or specialized operating system, such as a Unix, Windows™ or Linux™ based operating systems. The computer will comprise software, i.e. instructions coded on a non-transitory tangible computer-readable medium such as a memory drive or disk, which such instructions direct the calculations of model generation or predictive scoring. When all important values have been input to the processor, the predictive model will then calculate a predictive score indicative of the subject's PPTB risk, i.e. the subject's risk of experiencing PTB across subtypes±preeclampsia. This score may be retrieved from, transmitted from, displayed by or otherwise output by the computer. The computer can be specifically associated with a mass-spectrometer, ELISA reader, chip reader, or other chromatography equipement.

As is known in the art, the relative sensitivity and specificity of a predictive model can be adjusted to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about 0.7, at least about 0.75, at least about 0.8, at least about 0.85, at least about 0.9, or higher.

The raw data can be initially analyzed by measuring the values for each immune or growth-related biomarker, usually in triplicate or in multiple triplicates. The data can be manipulated, for example, raw data can be transformed using standard curves, and the average of triplicate measurements used to calculate the average and standard deviation for each patient. These values can be transformed before being used in the models, e.g. log-transformed, Box-Cox transformed (Box and Cox, Royal Stat. Soc., Series B, 26:211-246(1964). The data are then input into a predictive model, which will classify the sample. In some embodiments, the predicative data includes a plurality of values or ranges for each of a plurality of markers. The resulting information can be communicated to a patient or health care provider.

In one embodiment, hierarchical clustering is performed in the derivation of a predictive model, where the Pearson correlation is employed as the clustering metric. One approach is to consider a preterm birth dataset as a “learning sample” in a problem of “supervised learning.” CART is a standard in applications to medicine (Singer, Recursive Partitioning in the Health Sciences, Springer (1999)) and can be modified by transforming any qualitative features to quantitative features; sorting them by attained significance levels, evaluated by sample reuse methods for Hotelling's T²statistic; and suitable application of the lasso method. Problems in prediction are turned into problems in regression without losing sight of prediction, indeed by making suitable use of the Gini criterion for classification in evaluating the quality of regressions. This approach led to what is termed FlexTree (Huang, Proc. Nat. Acad. Sci. U.S.A 101:10529-10534(2004)).

FlexTree performs very well in simulations and when applied to multiple forms of data and is useful for practicing the claimed methods. Software automating FlexTree has been developed. Alternatively, LARTree or LART can be used (Turnbull (2005) Classification Trees with Subset Analysis Selection by the Lasso, Stanford University). The name reflects binary trees, as in CART and FlexTree; the lasso, as has been noted; and the implementation of the lasso through what is termed LARS by Efron et al. (2004) Annals of Statistics 32:407-451 (2004). See, also, Huang et al., Proc. Natl. Acad. Sci. USA. 101(29):10529-34 (2004). Other methods of analysis that can be used include logic regression. One method of logic regression Ruczinski, Journal of Computational and Graphical Statistics 12:475-512 (2003). Logic regression resembles CART in that its classifier can be displayed as a binary tree. It is different in that each node has Boolean statements about features that are more general than the simple “and” statements produced by CART.

Another approach is that of nearest shrunken centroids (Tibshirani, Proc. Natl. Acad. Sci. U.S.A 99:6567-72(2002)). The technology is k-means-like, but has the advantage that by shrinking cluster centers, one automatically selects features, as is the case in the lasso, to focus attention on small numbers of those that are informative. The approach is available as PAM software and is widely used. Two further sets of algorithms that can be used are random forests (Breiman, Machine Learning 45:5-32 (2001)) and MART (Hastie, The Elements of Statistical Learning, Springer (2001)). These two methods are known in the art as “committee methods,” that involve predictors that “vote” on outcome.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (Tusher et al., Proc. Natl. Acad. Sci. U.S.A 98, 5116-21 (2001)). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles. Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pair wise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

In an alternative analytical approach, variables chosen in the cross-sectional analysis are separately employed as predictors in a time-to-event analysis (survival analysis), where the event is the occurrence of preterm birth, and subjects with no event are considered censored at the time of giving birth. Given the specific pregnancy outcome (preterm birth event or no event), the random lengths of time each patient will be observed, and selection of proteomic and other features, a parametric approach to analyzing survival can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and corresponding functions are available with this model.

In addition, Cox models can be used, especially since reductions of numbers of covariates to manageable size with the lasso will significantly simplify the analysis, allowing the possibility of a nonparametric or semi-parametric approach to prediction of time to preterm birth. These statistical tools are known in the art and applicable to all manner of proteomic data. A set of immune- and growth-related biomarkers, clinical and genetic data that can be easily determined, and that is highly informative regarding the probability for preterm birth and predicted time to a preterm birth event in said pregnant female is provided. Also, algorithms provide information regarding the probability for preterm birth in the pregnant female.

In the development of a predictive model, it can be desirable to select a subset of markers, i.e., at least 3, at least 4, at least 5, at least 6, up to the complete set of markers. Usually a subset of markers will be chosen that provides for the needs of the quantitative sample analysis, e.g. availability of reagents, convenience of quantitation, etc., while maintaining a highly accurate predictive model. The selection of a number of informative markers for building classification models requires the definition of a performance metric and a user-defined threshold for producing a model with useful predictive ability based on this metric. For example, the performance metric can be the AUC, the sensitivity and/or specificity of the prediction as well as the overall accuracy of the prediction model.

As will be understood by those skilled in the art, an analytic classification process can use any one of a variety of statistical analytic methods to manipulate the quantitative data and provide for classification of the sample. Examples of useful methods include, without limitation, a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, partial least squares-discriminate analysis, multiple linear regression analysis, multivariate non-linear regression, backwards stepwise regression, threshold-based methods, tree-based methods, Pearson's correlation coefficient, Support Vector Machine, generalized additive models, supervised and unsupervised learning models, cluster analysis, or other predictive model known in the art.

In one embodiment, the disclosure provides a method of generating a predictive model to assess the risk for PTB across subtypes±preeclampsia in a pregnant female subject based on that subject's risk indicators. The predictive model is generated by a general process as follows: first, a panel of risk indicators is selected. Next, the risk indicator values for a first pool of women that experienced any form of PTB±preeclampsia during pregnancy, and the risk indicators for a. second pool of women did not experience any form of PTB±preeclampsia during pregnancy, are analyzed to derive mathematical relationships between risk indicator values and the probability of experiencing PTB across subtypes±preeclampsia.

The model may be derived from historical data sets comprising risk indicator values (e.g., maternal data and immune- and growth-related biomarker measurements) from a plurality of women in a population, wherein a subset of the women experienced any form PPTB±preeclampsia during pregnancy and another subset did not.

Various mathematical approaches exist for correlating multiple factors with the probability of a specified outcome. The predictive models of the disclosure may be generated using statistical methods such as: a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, partial least squares-discriminate analysis, multiple linear regression analysis, multivariate non-linear regression, backwards stepwise regression, threshold-based methods, tree-based methods, Pearson's correlation coefficient, Support Vector Machine, generalized additive models, supervised and unsupervised learning models, cluster analysis, or other predictive model known in the art. Subsets of the historical data may be utilized to generate, train, or validate the model, as known in the art.

The model input will comprise a risk indicator panel. The risk indicator panel may include measurements for immune- or growth-related biomarkers as described herein, and optionally, any additional secondary risk indicators, such as maternal characteristics, medical history, preexisting conditions (e.g., diabetes, hypertension, etc.), past pregnancy history, obstetrical history, and income status, or a subset thereof. For example, in one embodiment, the panel may comprise at least one risk indicator from each of the following categories: placental function, lipid status, hormonal status, and immune activity. Additional secondary risk indicators may be included as well, for example, race or ethnicity, income status, body weight, or body mass index, presence and/or severity of hypertension, diabetes, anemia, or other conditions, the stage of pregnancy, e.g. gestational age, and parity.

The model inputs may be expressed in various forms, for example being continuous variables, for example, the concentration of a particular immune- or growth-related biomarker in the serum of the subject. The input may comprise a median fluorescence intensity value. The model inputs may comprise normalized variables. For example, a subject's biomarker levels may be expressed as a multiple of the median value of a relevant population. The model inputs may also comprise categorical, discrete, and stratified values. For example, the existence of pre-existing diabetes comprises a discreet, yes or no value. In some embodiments, discreet variables may be assigned a numeric value, e.g. no=0 and yes=1. In another example, a biomarker level may be deemed elevated or not, by comparison to a reference value (e.g., an average population value or a value observed in subjects not at elevated risk for PTB across subtypes±preeclampsia). Likewise, a biomarker value can be assigned to a stratum (e.g., low, normal, or high).

The generated model will comprise one or more equations, into which an individual subject's risk indicator values may be inputted to generate an output that is predictive of that subject's risk for PTB across subtypes±preeclampsia. Model output may comprise a probability score, odds score, classifier score, risk categorical value (e.g. “low risk,” “moderate risk,” and “high risk,” etc.), such categories being based on statistical probabilities for PTB across subtypes±preeclampsia. The output may be further transformed to a probability, classification or other desired output based on methods known in the art. The output of the predictive model may be a score, which can be compared to one or more statistical cutoff values which define PTB across subtypes±preeclampsia risk categories.

To generate a predictive model for PTB across subtypes±preeclampsia, a predictive model was generated herein. Model 1 is a robust model that can predict the risk of PTB in pregnant subjects using a risk indicator panel comprising the twenty-five immune and growth-related biomarkers presented in Table 1, and two secondary risk indicators, i.e., the pregnant female subject being greater than 34 years of age and having a low-income status, see also Table 1. The predictive model is a linear discriminant analysis model with coefficients set forth in Table 1.

TABLE 1
Final 15-20 week linear discriminate for preterm birth (PTB) ± preeclampsia^{a}

| | No preterm | Preterm |
|---|---|---|
| | birth/PE | birth/PE |
| Constant | −2229 | −2207 |
| PAI1 (Uniprot accession number P05121) | 413.49597 | 411.87715 |
| Resistin (Uniprot accession number Q9HD89) | 0.75258 | 1.88708 |
| GP130 (Uniprot accession number Q13514) | 119.61108 | 118.44810 |
| ENA-78 (Uniprot accession number P42830) | −29.26997 | −28.53583 |
| sFASL (GenBank accession number P48023) | 5.54682 | 4.15190 |
| FGF-basic (Uniprot accession number P09038) | 200.03457 | 204.35713 |
| G-CSF (Uniprot accession number P09919) | 10.37429 | 10.68791 |
| IL-1R2 (Uniprot accession number P27930) | −2.50083 | −2.23721 |
| IL-4 (Uniprot accession number P05112) | −97.38072 | −94.75076 |
| IL-4R (Uniprot accession number P24394) | 23.32864 | 22.69110 |
| IL-5 (Uniprot accession number P05113) | 65.86996 | 63.28213 |
| IL-13 (Uniprot accession number P35225) | −35.04245 | −33.45918 |
| IL-17 (Uniprot accession number Q16552) | −114.44812 | −113.34045 |
| IL-17F (Uniprot accession number Q96PD4) | −1.80384 | −2.20769 |
| IFNB (Uniprot accession number P01574) | 4.26576 | 3.87186 |
| M-CSF (Uniprot accession number P09603) | −46.88392 | −47.52238 |
| NGF (Uniprot accession number P01138) | 8.44649 | 6.96815 |
| PDGFBB (Uniprot accession number E7FBB3) | −23.52635 | −22.59093 |
| RAGE (Uniprot accession number Q49A77) | −4.15909 | −3.75774 |
| SCF (Uniprot accession number Q13528) | 40.47520 | 37.72616 |
| VEGFR3 (Uniprot accession number P35916) | 14.01668 | 13.74962 |
| Eotaxin (Uniprot accession number P51671) | −51.73581 | −53.79304 |
| MIG (Uniprot accession number Q07325) | 5.47441 | 5.91727 |
| MIP1B (Uniprot accession number P13236) | 16.13980 | 14.87844 |
| RANTES (Uniprot accession number Q9UBL2) | 5.15387 | 4.74134 |
| Age >34 years | −15.30541 | −14.42951 |
| Low-income^{b} | 3.66412 | 4.71827 |

^{a}Results presented to the fifth decimal point to allow for complete transparency and replication of complete algorithm
^{b}Receiving assistance for medical services through the California MediCal program (requires an income of <138% of the federal poverty level)
Certain Accession Numbers are provided above, the data and sequences associated with each accession number are incorporated herein by reference for all purposes. Moreover, the accession numbers are exemplary, use of the UNIPROT or GENBANK websites will provide additional information associated with each accession number that can be used to characterize and describe the sequences etc. associated with each molecule.

The predictive model outputs a predictive PPTB classifier score for Subject X, as:

[PPTB risk Subject X]=(coefficient RI₁*measured value RI₁)+(coeefficient RI₂*measured value RI₂)+ . . . (coefficient RIₓ*measured value RIₓ)

wherein,

RI is a risk indicator or a secondary risk indicator as is described herein (e.g., see Table 1);

x is a number of 3 or greater;

coefficients are calculated by the methods described herein (e.g., see Table 1), with biomarker risk indicators are based upon log transformed biomarker serum concentration measurements as pg/mL; and secondary risk indicators are assigned Boolean values as follows: Subject using medical assistance=1, subject not using medical assistance=0 and subject age>34=1, subject age<34=0, etc.

The output of the discriminant function can be a classifier indicating that the subject is at risk for PPTB or not. The output of the discriminant function can be converted to a probability or other risk score by a statistical means described herein or known in the art. An “elevated” risk of PPTB can be selected based on desired criteria, for example, a 10-99% risk may be deemed elevated depending on context.

In testing against historical data, the predictive models described herein accurately predicted the risk of PPTB in subjects experiencing spontaneous PTB, induced PTB, and preeclampsia, as is summarized in Table 5.

In various implementations of the predictive models, one or more of the coefficients may be adjusted upwards or downwards by at least 1%, 2%, 3%, 4%, 5%, 6-10%, or 10-15%, or more.

The studies presented herein focused on the capacity for prediction of PTB±preeclampsia. It was found that the serum markers that have established links with poor pregnancy outcomes and close ties to immune function and growth provide good insight into pathophysiological underpinnings of PTB. Most notably, the findings from the studies presented herein are supportive of the role of perturbation of the cytokine network in the pathogenesis of PTB. The effectiveness of the methods disclosed herein in prediction of PTB was driven by a constellation of markers that were often highly related yet contributed independently to prediction. The study data also found that combining cross-pathway markers increases the predictive performance of the methods of the disclosure. By combining cross-way molecular markers with risks like maternal age >34 years and low-income status, the methods and models presented herein took advantage of maternal risks for PTB along with important pathway signals.

The studies presented herein indicated a strong association between PTB and low-income status (including when defined by participation in state-sponsored health insurance programs for individuals with incomes near or below the United States poverty line). It was suspected that low income status was serving as a proxy for unmeasured or underreported factors with links to PTB±preeclampsia including, possibly, the presence of nutritional deficits, psycho-social or systemic stress, and greater exposure to potentially harmful substances like tobacco, alcohol, and pollution. While there was information about tobacco and alcohol use (as well as drug use) in the study dataset, it is possible that these factors were underreported and as such, that low income status is serving as a proxy for these factors as well as others that may be more common with poverty. It is important to note that in the present study these factors alone were poor predictors of preterm birth (with AUCs below 62% in the training and testing sets) and also that they contributed a relatively small amount of information over and above biomarkers alone (increasing the AUC for biomarkers only by 0.026±0.058 in the training set and by 0.008±0.075 in the testing set). As such, it is clear that these factors alone were not the sole drivers of overall risk and may point to more upstream drivers. Nevertheless, it is important to investigate these patterns more completely given potential for modification. Accordingly, in certain embodiments presented herein, the method of the disclosure further comprises secondary test factors, including, but not limited to, the income status of the test subject, drug use, and tobacco and alcohol use. These data also suggest that the efficacy of the methods disclosed herein would not be diminished in settings characterized by mostly high- or low-income individuals given that molecular factors appear to be the primary drivers of prediction.

In view of the results presented herein, the methods of the disclosure represent an improvement over other tests for PTB±preeclampsia, particularly given applicability across PTB subgroups and to larger populations given the use of a random sampling design and the leveraging of multiplex technology available globally. Given that the methods described herein performed well with samples collected at as early as 15-weeks of gestation, there is high confidence that the methods of the disclosure could be applied at earlier gestational ages, in particular 16-weeks of gestation when aspirin administration has the greatest efficacy in preventing preeclampsia. Given mounting data demonstrating that early term babies are at increased risk for both short- and long-term morbidity and that these women are more likely to deliver preterm in the next pregnancy it would be advantageous to be able to identify these women early in pregnancy in an effort to extend gestation.

The full LDA function used for classification in the methods disclosed herein have been provided (see Table 1) so that the methods of the disclosure can be carried out in a variety of testing settings. Some of the markers in the studies—namely FGF-basic and IL-4 exhibited a particularly large influence on the PTB±preeclampsia algorithm while also having large observed confidence intervals in initial multivariate logistic models (see Table 4). Both of these factors were normally distributed after log transformation and as such, the large risks and confidence intervals observed appeared to be driven by the separation of values for these markers in cases vs. controls after adjustment for the other factors in the methods of disclosure. Given this and the contribution of both to AUC performance these factors in certain embodiments can be used in the methods disclosed herein. In addition, it should be recognized that because many of the markers in the model are highly correlating, but were retained due to their individual contribution to the c-statistic. As such, in other embodiments, of the disclosure the methods disclosed herein may optionally comprise these markers.

In additional embodiments, the predictability of the methods disclosed herein can be greatly enhanced by consideration of additional risk indicators, such as maternal factors, like maternal age and poverty status. Thus, in particular embodiments, the methods of the disclosure further comprise evaluation of risk indicators, such as maternal factors, like maternal age and poverty status. In summary, along with maternal age and poverty status, mid pregnancy immune and growth factors measured by the methods of the disclosure reliably identified women who went on to have a PTB±preeclampsia. Accordingly, the methods disclosed herein have the potential to be used to identify women who may benefit from existing and emerging interventions aimed at reducing rates of PTB and preeclampsia.

Furthermore, the methods and biomarker panels of the disclosure can be applied in various ways:

For example, the methods and biomarker panels can be used to calculate or asses the risk of pregnant female for PTB across subtypes±preeclampsia by providing a risk score or risk assessment, and can include steps such as,

measuring the levels of immune- and/or growth-related biomarkers as described herein, or panels thereof, and optionally secondary risk indicators, from a biological sample obtained from a subject;

assigning risk indicator values for each of the measured immune- and/or growth-related biomarkers or panels thereof (and secondary risk indicators, if included);

inputting the obtained risk indicator values to a predictive model based on the selected panel of immune- and/or growth-related biomarkers (and secondary risk indicator, if included); and

calculating a PPTB risk assessment for the subject using the predictive model.

The methods can further provide steps for prophylactically administering a therapy to the subject, if the subject is found to have increased risk for PTB across subtypes±preeclampsia, e.g., by having a certain risk score or assessment. In a further embodiment, the selection of the intervention is guided by the risk indicator profile used to assess the subject's risk for PTB across subtypes±preeclampsia.

The acquisition of risk indicators for PTB across subtypes±preeclampsia values, e.g., can be by measuring the levels of one or more immune- or growth-related biomarker described herein, or panels thereof, and for secondary risk indicators, by obtaining medical records, running medical tests, measuring physical characteristics of the subject (e.g., height, weight, blood pressure, BMI, etc.), interviewing the subject, having the subject fill out questionnaires, etc. This step can be performed by one or more practitioners in one or more separate operations. Missing values may be accounted for using statistical tools known in the art.

For biomarkers assessment, the immune- or growth-related biomarkers disclosed herein may be quantified in a suitable biological sample obtained from the subject, such as a serum sample. Quantification of biomarkers in samples may be performed by any using the methods already disclosed herein, or other methods known in the art. In a particular embodiment, a multiplex immunoassay is utilized to measure one or more, or all, of the immune- or growth-related biomarkers described herein. For example, a multiplex bead immunoassay may be utilized, wherein sets of uniquely labeled and identifiable beads, each uniquely labeled bead targeted to a single biomarker target, are used to simultaneously assay a sample for a panel of biomarkers. Exemplary multiplex assay platforms include those described in U.S. Pat. No. 8,075,854, entitled “Microfluidic chips for rapid multiplex ELISA,” by Yang; United States Patent Publication Number US20020127740, entitled “Quantitative microfluidic biochip and method of use,” by Ho, and United States Patent Publication Number 20040241776, entitled “Multiplex enzyme-linked immunosorbent assay for detecting multiple analytes,” by Giester. An exemplary multiplex immunoassay is the Luminex XMAP™ or like system. Mass spectrometry techniques may be utilized to analyze biomarker presence and/or concentration in the sample. For example, MALDI or SELDI mass spectroscopy techniques can be employed, as known in the art. Other analytical approaches as described herein, can be used as well.

The attained risk indicator values for each of the immune- or growth-related biomarkers, or a panel thereof, and optionally risk indicator values for secondary risk indicators, are then inputted to the predictive model. The predictive model may comprise any model based on the selected risk indicators, for example, a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, partial least squares-discriminate analysis, multiple linear regression analysis, multivariate non-linear regression, backwards stepwise regression, threshold-based methods, tree-based methods, Pearson's correlation coefficient, Support Vector Machine, generalized additive models, supervised and unsupervised learning models, cluster analysis, or other predictive model known in the art.

The predictive calculations of the model (as well as model generation steps described in the previous section) may be carried out by any suitable digital computer. Suitable digital computers may include portable devices, laptop and desktop computers, cloud computing systems, etc., using any standard or specialized operating system, such as a Unix, Windows™ or Linux™ based operating systems. The computer will comprise software, i.e. instructions coded on a non-transitory tangible computer-readable medium such as a memory drive or disk, which such instructions direct the calculations of model generation or predictive scoring.

When the values have been inputted to the processor, the predictive model will then calculate a risk score indicative of the subject's risk of experiencing one or more of PTB (by any form)±preeclampsia. This risk score may be retrieved from, transmitted from, displayed by or otherwise outputted by the computer.

As described herein, the immune- and growth-related biomarkers described herein, as well as the secondary risk indicators, are highly predictive of a subject's risk for PTB (by any form)±preeclampsia. Accordingly, the disclosure further provides for integrated assays to simultaneously measure multiple PPTB risk indicators in a single sample, such as assay kits. The assay kits described herein can be used to assess the levels of the immune- and growth-related biomarkers disclosed herein that have been shown to have a high correlation for PTB (by any form)±preeclampsia. Such assay kits provide a “one stop” kit to assess the relevant PPTB associated biomarkers in a biological sample, so that a risk assessment of the subject for PTB (by any form)±preeclampsia is convenient and easily to quantify/assess. In a particular embodiment, the kit comprises, consists essentially of, or consists of the 25 immune- and growth-related biomarkers described in Table 1. In another embodiment, the kit is directed to the quantification of a subset of the 25 immune- and growth-related biomarkers described in Table 1.

The assay kit will comprise a plurality of detection/quantification tools specific to each biomarker detected by the kit. Many of the biomarkers disclosed herein comprise proteins, which may be detected by immunoassays or like technologies. The detection/quantification tools may comprise capture ligands of multiple types, each directed to the selective capture of a specific biomarker in the sample. The detection/quantification tools may comprise labeling ligands of multiple types, each directed to the selective labeling of a specific biomarker in the sample, for example, comprising enzymatic, fluorescent, or chemiluminescent labels for the quantification of target species. For example, the capture and/or labeling ligands may comprise antibodies (or fragments thereof), affibodies, aptamers, or other moieties that specifically bind to a selected biomarker. The assay kit may further comprise labeled secondary antibodies, for example comprising enzymatic, fluorescent, or chemiluminescent labels and associated reagents.

In one embodiment, the assay kit comprises a solid support to which one or more individually addressable patches of capture ligands are present, wherein the capture ligands of each patch are directed to a specific immune or growth-related biomarker described herein. In another embodiment, individually addressable patches of absorbent or adsorbing material are present, onto which individual aliquots of sample may be immobilized. Solid supports may include, for example, a chip, wells of a microtiter plate, a bead or resin. The chip or plate of the kit may comprise a chip configured for automated reading, as is known in the art.

In another embodiment, the assay kits of the disclosure are SELDI probes comprising capture ligands present on a solid support, which can capture the selected biomarkers from the sample and release them in response to a desorption treatment for mass spectroscopic analysis.

In yet another embodiment, the assay kits of the disclosure comprise reagents or enzymes which create quantifiable signals based on concentration dependent reactions with biomarker species in the sample. Assay kits may further comprise elements such as reference standards of the biomarkers to be measured, washing solutions, buffering solutions, reagents, printed instructions for use, and containers.

The following examples are intended to illustrate but not limit the disclosure. While they are typical of those that might be used, other procedures known to those skilled in the art may alternatively be used.

### EXAMPLES

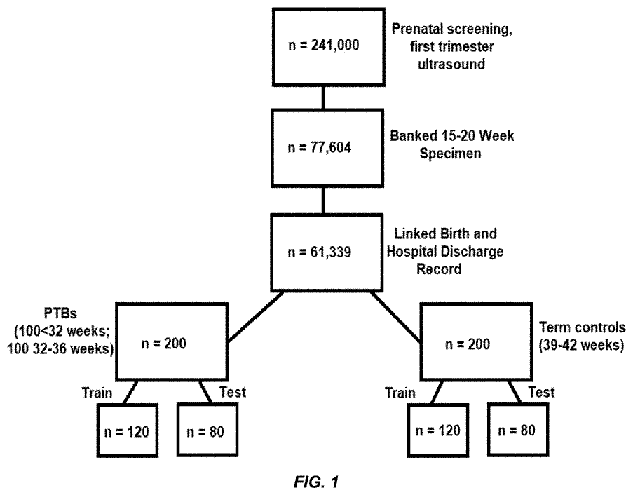

Materials and Methods: All women included in the study are part of a population based cohort of all singleton California births from July 2009 through December 2010 (n=757,853). All women had gestational dating by first trimester ultrasound and had a second trimester serum marker test done as part of routine prenatal screening for aneuploidies and neural tube defects by the California Genetic Disease Screening Program (n=241,000). Candidate cases and controls all had a second trimester serum sample banked by the California Biobank Program (n=77,604) and had detailed demographic and obstetric information available in a linked hospital discharge birth cohort database maintained by the California Office of Statewide Health Planning and Development (OSHPD) (n=61,339). A number of previous papers have been published that leverage data and screening results for women in this and other California cohorts. The final source set for this study included 4025 singletons with births before 37 weeks, and 56,081 with births on or after 37 completed weeks through 44 weeks. From this set, 100 PTB cases were selected with gestational ages at birth <32 weeks, 100 PTB cases with gestational ages at birth from 32 to 36 weeks, and 200 term controls with gestational ages at birth from 39 to 42 weeks using simple random sampling wherein each within group pregnancy had an equal probability of selection. The resulting sample (by <32, 32-26, and 39 to 42 weeks) were then divided into training and testing subsets at a ratio of 2:1 (see FIG. 1). This was a convenient random sample wherein total number was determined based on the financial resources available for testing.

Maternal demographic and obstetric characteristics. Demographic and obstetric factors evaluated included race/ethnicity, maternal age, years of formal education, place of maternal birth, low-income status (as indicated by “Medi-Cal” payment for delivery (the California health program for low-income persons (generally defined as income <138% of the United States poverty level)), parity, preexisting diabetes, preexisting hypertension, reported smoking, obesity (body mass index (BMI) ≥30 m/kg²), interpregnancy interval (IPI) <12 months, and previous PTB. All variables were derived from the OSHPD birth cohort file, which combines birth certificate records and all hospital discharge records for the mother and baby from 1 year prior to the birth to 1 year after the birth. Coding of preexisting and gestational diabetes and hypertension was based on International Classification of Diseases, 9′ Revision, Clinical Modification (ICD-9-CM) four digit codes contained in the cohort file.

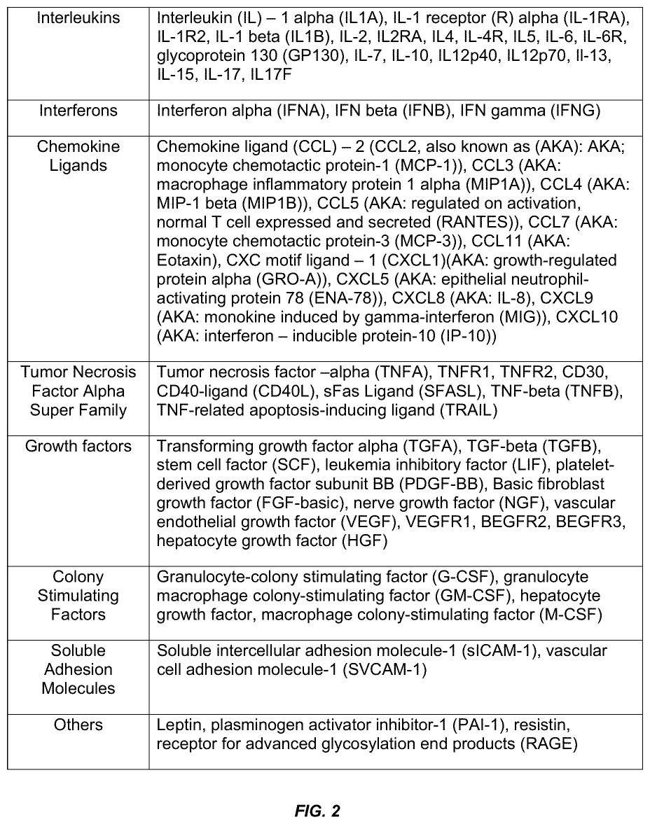

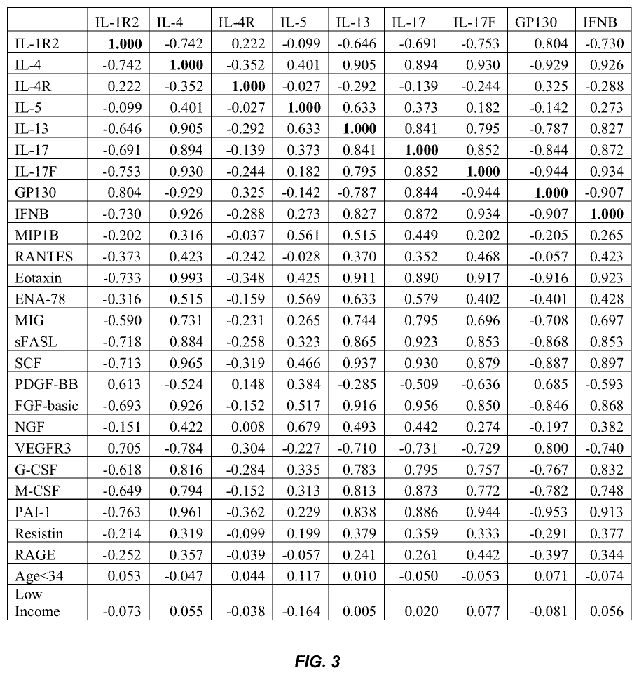

Serum biomarker testing. Immune and growth-factor molecular testing was done using residual serum samples from second trimester (15-20 week) prenatal screening. Specimens were stored in 1 milliliter tubes at −80 ° C. Markers tested included twenty interleukins, three interferons, eleven chemokine ligands, eight members of the tumor necrosis factor-alpha (TNFA) super family cytokines, 12 growth factors, three colony stimulating factors, two soluble adhesion molecules, and leptin, plasminogen activator inhibitor-1 (PAI-1), resistin, and receptor for advanced glycosylation end products (RAGE) (see FIG. 2 for complete listing). While many of these markers have been shown to have close links to PTB or preeclampsia, the full panel of immune and growth-factor related markers available were evaluated via multiplex testing at the Human Immune Monitoring Center (HIMC) at Stanford University for this study. Based upon the established interconnectedness of all of these markers to immune function and as such, there was potential for revealing novel patterns and relationships—particularly given the role of immune function in pregnancy.

All markers were read using a Luminex 200 instrument (Austin, Tex.) in accordance with the manufacturer recommendations. All markers were tested using a human multiplex kit that was purchased from Affymetrix Inc. (Santa Clara, Calfi.) with the exception of human soluble receptors, which were measured using a Millipore high sensitivity multiplex kit (HSCRMAG32KPX14) (Billerica, Mass.). Median fluorescence intensity (MFI) values were reported for all markers using Masterplex software (Hitashi Solutions, San Bruno, Calif.). To avoid error inherent in log transformation of MFI to pg/mL, analyses relied on the MFI average, which was based on measurement of two aliquots tested on the same plate for each case and control. All inter-assay coefficients (CVs) were <15% across all markers and all intra-assay CVs were <10%.

Data analyses. Simple logistic regression (including odds ratios (ORs) and their 95% (CIs)) were used for association testing in the training set using demographic, clinical, and molecular factors (standardized using natural log transformation) and to build multivariate models. So as not to lose information that might be important to prediction, for variable selection into multivariate models backward stepwise regression was utilized wherein all possible predictors were entered into the model and the criteria for remaining in the model was p<0.20. Predictors with a p≥0.05 and <0.20 were removed in any instance where their exclusion resulted in a <1% decrease in the concordance statistic (cstatistic) (equivalent to the area under the receiver operating characteristic curve (AUC)). Similarly, in any instance where the variable inflation factor (VIF) indicated major multicollinearity among predictors (defined as VIF≥2.5) predictors were removed when their exclusion resulted in a <1% decrease in the c-statistic. All variables in the final multivariate logistic model were included in the final linear discriminate analysis (LDA) algorithm with assessment of performance using AUC in both the training and testing subsets. AUC performance was evaluated for all PTBs and for early PTB (<32 weeks) and late PTB (33-36) subgroups including in spontaneous and provider initiated subgroups and by preeclampsia diagnosis by ICD-9-CM code. “Spontaneous PTBs” were considered to be those where the birth certificate or hospital discharge record noted “preterm premature rupture of membranes” (PPROM) or “preterm labor.” Pregnancies with a record of receiving tocolytics with no record of PPROM were also included in the preterm labor group. Pregnancies classified as “provider initiated” PTB were those without PPROM or premature labor for whom there was “medical induction”, “assisted rupture of membranes”, or for whom there was a cesarean delivery at <37 weeks of gestation and none of the aforementioned indicators of spontaneous PTB. Rates of PTB (overall and by subtypes and by preeclampsia) were examined by AUC derived probability scores (by deciles) to assess true- and false-positive performance at set cut-points in the training and testing subgroups.

All analyses were done using Statistical Analysis Software (SAS) version 9.3 (Cary, N.C.). Methods and protocols for the study were approved by the Committee for the Protection of Human Subjects within the Health and Human Services Agency of the State of California, the Institutional Review Board of Stanford University and the Institutional Review Board of the University of California San Francisco.

Results. Most case and control women in the study identified themselves as Hispanic or White (e.g., 55.8% of women with a PTB delivery and 42.5% of women with a term delivery in the training sample were Hispanic and 47.5% of women with a PTB delivery and 42.5% of women with a term delivery in the testing sample were Hispanic). Most women in both the training and testing samples were between 18 and 34 years of age (67.5-75.0% across groupings). The majority women with a preterm delivery had a spontaneous PTB (82.5% in the training sample and 75.0% in the testing sample). The rate of preterm preeclampsia was 15.8% in the training sample and 22.5% in the testing sample (see Table 2).

TABLE 2
Sample characteristics

| | Training | | Testing | |
|---|---|---|---|---|
| | PTB n (%) | Term n (%) | PTB n (%) | Term n (%) |
| Sample | 120 (100.0) | 120 (100.0) | 80 (100.0) | 80 (100.0) |
| Race/ethnicity | | | | |
| Hispanic | 67 (55.8) | 51 (42.5) | 38 (47.5) | 34 (42.5) |
| White | 39 (32.5) | 49 (40.8) | 26 (32.5) | 35 (43.8) |
| Asian | 8 (6.7) | 9 (7.5) | 11 (13.8) | 5 (6.3) |
| Black | 3 (2.5) | 3 (2.5) | 3 (3.8 ) | 1 (1.3) |
| Other | 0 | 1 (0.8) | 2 (2.5) | 0 |
| Age (Years) | | | | |
| <18 | 1 (0.8) | 2 (1.7) | 1 (1.3) | 0 |
| 18-34 | 81 (67.5) | 90 (75.0) | 56 (70.0) | 59 (73.8) |
| ≥35 | 38 (31.7) | 28 (23.3) | 23 (28.8) | 21 (26.3) |
| Other (all | | | | |
| yes vs. no) | | | | |
| <12 years | 22 (18.3) | 21 (17.5) | 16 (20.0) | 11 (13.8) |
| education | | | | |
| Born in the | 76 (63.3) | 85 (70.8) | 50 (62.5) | 54 (67.5) |
| United States | | | | |
| Low-Income^{a} | 61 (50.8) | 40 (33.3) | 35 (43.8) | 30 (37.5) |
| Nulliparous | 54 (45.0) | 64 (53.3) | 40 (50.0) | 39 (48.8) |
| Reported | 3 (2.5) | 2 (1.7) | 1 (1.3) | 1 (1.3) |
| smoking | | | | |
| Obese | 29 (24.2) | 21 (17.5) | 18 (22.5) | 10 (12.5) |
| Preexisting | 3 (2.5) | 1 (0.8) | 4 (5.0) | 1 (1.3) |
| diabetes | | | | |
| Preexisting | 7 (5.8) | 3 (2.5) | 10 (12.5) | 0 |
| hypertension | | | | |
| Anemia | 8 (6.7) | 12 (10.0) | 11 (13.8) | 2 (2.5) |
| IPI | 24 (20.0) | 28 (23.3) | 13 (16.3) | 14 (17.5) |
| <12 Months | | | | |
| Preterm birth | | | | |
| subgroups | | | | |
| Spontaneous | 99 (82.5) | | 60 (75.0) | |
| Provider | 17 (14.2) | | 18 (22.5) | |
| initiated | | | | |
| Subtype | 4 (3.3) | | 2 (2.5) | |
| unknown | | | | |
| <32 Weeks | 60 (50.0) | | 40 (50.0) | |
| Spontaneous | 53 (44.2) | | 32 (40.0) | |
| Provider | 5 (4.2) | | 8 (10.0) | |
| initiated | | | | |
| Subtype | 2 (1.7) | | 2 (2.5) | |
| unknown | | | | |
| 32-36 Weeks | 60 (50.0) | | 40 (50.0) | |
| Spontaneous | 46 (38.3) | | 28 (35.0) | |
| Provider | 12 (10.0) | | 10 (12.5) | |
| initiated | | | | |
| Subtype | 2 (1.7) | | 2 (2.5) | |
| unknown | | | | |
| Preeclampsia | 19 (15.8) | 2 (1.7) | 18 (22.5) | 1 (1.3) |
| (any) | | | | |
| <32 Weeks | 9 (7.5) | | 13 (16.3) | |
| 32-36 Weeks | 10 (8.3) | | 5 (6.3) | |

IPI interpregnancy interval
^{a}Receiving assistance for medical services through the California MediCal program (requires an income of <138% of federal poverty level)

Crude logistic analyses in the training sample revealed that women with PTB±preeclampsia were significantly more likely (p <0.05) than term controls to be low-income (as indicated by MediCal status) (OR 2.07, 95% CI 1.23-3.48) and to have lower MIP1B levels (OR 0.59, 95% CI 0.38-0.93) (see Table 3).

TABLE 3
Crude odds ratios, training set: Demographic, clinical, and
serum biomarkers in term births versus preterm births ±
preeclampsia (all serum markers log transformed).

| | Odds Ratio | 95% CI | P= |
|---|---|---|---|
| Race/ethnicity^{a} | | | |
| Hispanic | 1.65 | 0.95-2.88 | 0.08 |
| Asian | 1.12 | 0.39-3.16 | 0.83 |
| Black | 1.26 | 0.24-6.57 | 0.79 |
| Age (Years)^{b} | | | |
| <18 | 0.56 | 0.05-6.24 | 0.63 |
| ≥35 | 1.50 | 0.85-2.66 | 0.16 |
| Other^{c} | | | |
| <12 Years Education | 1.06 | 0.55-2.05 | 0.87 |
| Born in the United States | 0.71 | 0.41-1.22 | 0.22 |
| Low Income^{d} | 2.07 | 1.23-3.48 | <0.01 |
| Nulliparous | 0.72 | 0.43-1.19 | 0.20 |
| Reported Smoking | 1.51 | 0.25-9.22 | 0.65 |
| Obese | 1.50 | 0.80-2.82 | 0.21 |
| Preexisting Diabetes | 3.05 | 0.31-29.76 | 0.34 |
| Preexisting Hypertension | 2.42 | 0.61-9.57 | 0.21 |
| Anemia | 0.64 | 0.25-1.63 | 0.35 |
| IPI <12 Months | 0.82 | 0.44-1.52 | 0.53 |
| Interleukins^{e} | | | |
| IL-1A | 0.95 | 0.70-1.27 | 0.71 |
| IL-1RA | 0.88 | 0.58-1.33 | 0.53 |
| IL-1R2 | 1.02 | 0.82-1.27 | 0.87 |
| IL-1B | 1.00 | 0.88-1.13 | 0.99 |
| IL-2 | 1.00 | 0.78-1.29 | 0.99 |
| IL-2RA | 0.89 | 0.58-1.37 | 0.59 |
| IL-4 | 1.02 | 0.85-1.23 | 0.82 |
| IL-4R | 0.82 | 0.55-1.21 | 0.31 |
| IL-5 | 0.67 | 0.37-1.20 | 0.18 |
| IL-6 | 0.83 | 0.58-1.17 | 0.28 |
| IL6R | 0.72 | 0.38-1.38 | 0.32 |
| GP130 | 0.94 | 0.79-1.12 | 0.46 |
| IL-7 | 0.77 | 0.45-1.32 | 0.34 |
| IL-10 | 0.99 | 0.79-1.25 | 0.96 |
| IL-12p40 | 0.96 | 0.80-1.14 | 0.62 |
| IL-12p70 | 0.72 | 0.39-1.34 | 0.30 |
| IL-13 | 0.95 | 0.69-1.31 | 0.77 |
| IL-15 | 0.98 | 0.72-1.33 | 0.88 |
| IL-17 | 0.99 | 0.75-1.31 | 0.95 |
| IL17F | 1.00 | 0.90-1.11 | 0.99 |
| Interferons^{e} | | | |
| IFNA | 0.99 | 0.87-1.12 | 0.83 |
| IFNB | 0.99 | 0.87-1.13 | 0.88 |
| IFNG | 1.01 | 0.90-1.13 | 0.88 |
| Chemokine Ligands^{e} | | | |
| MCP1 | 1.02 | 0.87-1.20 | 0.78 |
| MIP1A | 0.90 | 0.77-1.05 | 0.17 |
| MIP1B | 0.59 | 0.38-0.93 | 0.02 |
| RANTES | 0.91 | 0.71-1.18 | 0.47 |
| MCP3 | 0.98 | 0.79-1.22 | 0.86 |
| Eotaxin | 1.01 | 0.82-1.24 | 0.93 |
| GRO-A | 1.01 | 0.88-1.15 | 0.90 |
| ENA-78 | 1.00 | 0.71-1.41 | 0.98 |
| IL-8 | 1.02 | 0.90-1.16 | 0.73 |
| MIG | 1.06 | 0.90-1.25 | 0.52 |
| IP-10 | 1.01 | 0.72-1.41 | 0.95 |
| Tumor Necrosis Factor | | | |
| Alpha Super Family^{e} | | | |
| TNFA | 0.97 | 0.79-1.21 | 0.80 |
| TNFR1 | 0.70 | 0.40-1.21 | 0.20 |
| TNFR2 | 0.86 | 0.40-1.84 | 0.69 |
| CD30 | 1.01 | 0.73-1.40 | 0.95 |
| CD40L | 0.82 | 0.62-1.08 | 0.16 |
| sFASL | 0.96 | 0.79-1.18 | 0.72 |
| TNFB | 0.99 | 0.85-1.15 | 0.85 |
| TRAIL | 0.87 | 0.60-1.28 | 0.49 |
| Growth Factors^{e} | | | |
| TGFA | 0.97 | 0.79-1.21 | 0.80 |
| TGFB | 1.03 | 0.84-1.26 | 0.79 |
| SCF | 0.97 | 0.75-1.25 | 0.82 |
| LIF | 1.02 | 0.85-1.23 | 0.82 |
| PDGFBB | 0.87 | 0.63-1.20 | 0.39 |
| FGF-Basic | 1.01 | 0.71-1.44 | 0.97 |
| NGF | 0.47 | 0.21-1.05 | 0.07 |
| VEGF | 0.95 | 0.66-1.35 | 0.76 |
| VEGFR1 | 0.97 | 0.88-1.08 | 0.61 |
| VEGFR2 | 0.95 | 0.82-1.09 | 0.45 |
| VEGFR3 | 0.96 | 0.83-1.12 | 0.63 |
| HGF | 1.00 | 0.79-1.27 | 0.99 |
| Colony Stimulating | | | |
| Factors^{e} | | | |
| G-CSF | 1.06 | 0.89-1.27 | 0.57 |
| GM-CSF | 0.93 | 0.67-1.30 | 0.67 |
| M-CSF | 0.97 | 0.79-1.19 | 0.77 |
| Soluble Adhesion | | | |
| Molecules^{e} | | | |
| sICAM1 | 0.90 | 0.70-1.15 | 0.38 |
| sVCAM1 | 1.15 | 0.86-1.54 | 0.35 |
| Others | | | |
| Leptin | 0.84 | 0.63-1.12 | 0.23 |
| PAI1 | 1.02 | 0.75-1.38 | 0.91 |
| Resistin | 1.11 | 0.73-1.70 | 0.63 |
| RAGE | 1.16 | 0.82-1.65 | 0.41 |

CI, Confidence interval
^{a}Odds ratio computed with White race/ethnicity as referent.
^{b}Odds ratio computed with 18-34 years of age as referent.
^{c}Odds ratio computed as yes versus no.
^{d}Receiving assistance for medical services through the California MediCal program (requires an income of <138% of the federal poverty level).
^{e}See FIG. 2 for full biomarker names.

The final 15 to 20-week PTB±preeclampsia model included maternal age greater than 34-years and low-income status along with 25 serum biomarkers (see Table 4).

TABLE 4
Markers from multivariate logistic model
included in final linear discriminate for
preterm birth ± preeclampsia.

| | Odds Ratio | 95% CI | p= |
|---|---|---|---|
| PAI1 | 0.14 | 0.01-1.62 | 0.12^{c} |
| Resistin | 3.15 | 1.53-6.48 | 0.01 |
| GP130 | 0.29 | 0.10-0.82 | 0.02 |
| ENA-78 | 2.12 | 1.06-4.24 | 0.04 |
| sFASL | 0.21 | 0.06-0.70 | 0.01 |
| FGF-Basic | 66.74 | 3.02->999.99 | 0.01 |
| G-CSF | 1.48 | 0.81-2.70 | 0.19^{c} |
| IL-1R2 | 1.40 | 0.87-2.25 | 0.17^{c} |
| IL-4 | 15.68 | 1.04-236.90 | 0.05 |
| IL-4R | 0.55 | 0.27-1.12 | 0.10^{c} |
| IL-5 | 0.09 | 0.01-1.17 | 0.07^{c} |
| IL-13 | 6.57 | 0.91-47.67 | 0.06^{c} |
| IL-17 | 5.28 | 0.73-37.93 | 0.10^{c} |
| IL-17F | 0.67 | 0.38-1.19 | 0.17^{c} |
| IFNB | 0.67 | 0.36-1.23 | 0.20^{c} |
| M-CSF | 0.47 | 0.18-1.21 | 0.12^{c} |
| NGF | 0.08 | 0.01-0.96 | 0.05 |
| PDGFBB | 3.01 | 1.23-7.39 | 0.02 |
| RAGE | 1.63 | 0.94-2.82 | 0.08^{c} |
| SCF | 0.05 | 0.01-0.43 | 0.01 |
| VEGFR3 | 0.70 | 0.47-1.04 | 0.08^{c} |
| Eotaxin | 0.13 | 0.01-2.18 | 0.16^{c} |
| MIG | 1.64 | 1.00-2.68 | 0.05 |
| RANTES | 0.62 | 0.38-1.02 | 0.06^{c} |
| Age >34 Years | 2.58 | 1.24-5.36 | 0.01 |
| Low Income^{b} | 2.80 | 1.44-5.45 | <0.01 |

^{a}For logistic model there were no p-value limits on entry, retention at p ≤ .20 with further exclusion where decrease in area under the Receiver Operating Characteristic curve (AUC) was <1.0%.
^{b}Receiving assistance for medical services through the California MediCal program (requires an income of <138% of federal poverty level).
^{c}Factor included in model despite p > .05 given that removal resulted in a ≥1.0% decrease in AUC.

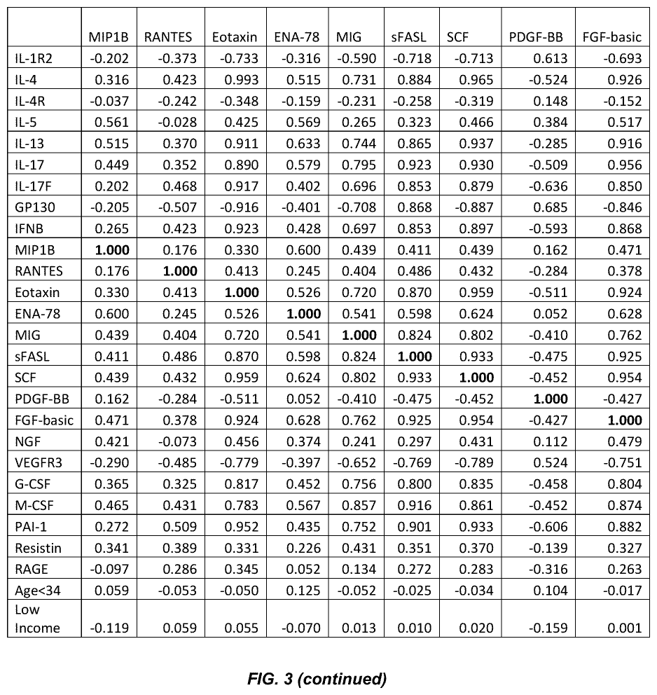

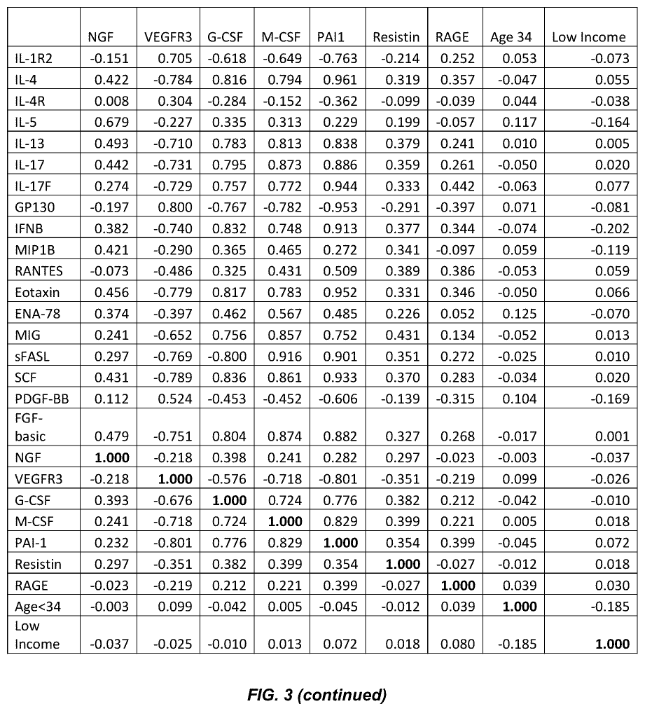

Serum markers included eight interleukins (IL-1 receptor 2 (IL-1R2), IL-4, IL-4R, IL-5, IL-13, IL-17, IL-17F, and glycoprotein 130 (GP130)), one interferon (interferon (IFN) beta (IFNB)), one factor from the TNFA super family (sFAS ligand (sFASL)), five chemokine ligands (epithelial neutrophil-activating protein 78 (ENA-78), eotaxin, monokine induced by gamma-interferon (MIG), macrophage inflammatory protein 1 beta (MIP1B), and regulated on activation, normal T-cell expressed and secreted (RANTES)), five growth factors (stem cell factor (SCF), platelet-derived growth factor subunit BB (PDGFBB), basic fibroblast growth factor (FGF-basic), nerve growth factor (NGF), and vascular endothelial growth factor R3 (VEGFR3)), two colony-stimulating factors (granulocyte-colony-stimulating factor (G-CSF), and macrophage colony-stimulating factor (M-CSF)), as well as PAI1, resistin, and RAGE. Although we found that many of the markers in the final model were highly correlated (VIFs≥2.5 for 21 of the 24 markers in the final model (IL-1R2, IL-4, IL-5, IL-13, IL-17, IL-17F, GP130, IFNB, sFASL, ENA-78, eotaxin, MIG, MIP1B, SCF, PDGF-BB, FGF-basic, NGF, VEGFR3, G-CSF, M-CSF, and PAI1) (see FIG. 3), all of these markers contributed 1% or more to the c-statistic when included in the model and were, therefore, retained.

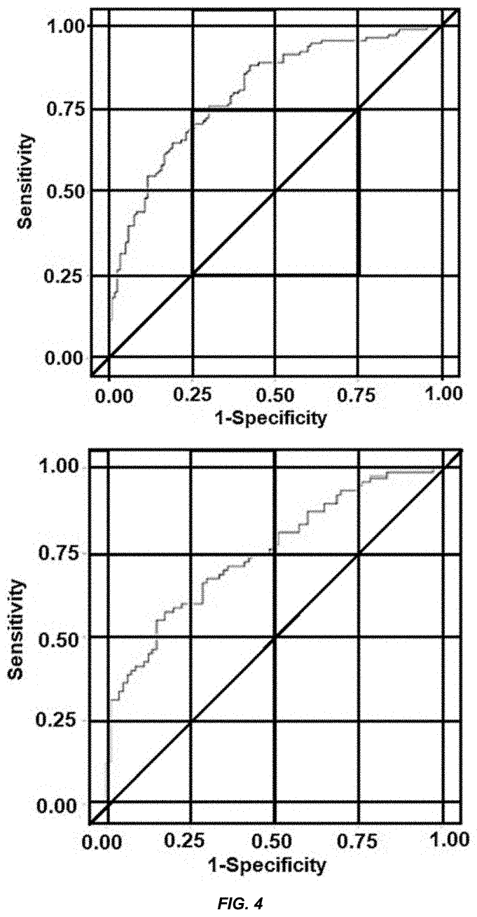

When considered in combination using the linear discriminate for PTB±preeclampsia, the 25-target immune and growth factors along with maternal age >34 years and low-income status were able to identify more than 80% of women going on to deliver preterm in the training set (AUC 0.803, 95% CI 0.748-0.858) and 75.0% of women going on to deliver preterm in the testing set (AUC 0.750, 95% CI 0.676-0.825) (see Table 5, see also FIG. 4).

TABLE 5
Performance of mid-pregnancy immune and growth
factor preterm birth ± preeclampsia test (overall and
by preterm and preeclampsia subgroups)

| | Training (n = 240) | | Testing (n = 160) | |
|---|---|---|---|---|
| | AUC | 95% CI | AUC | 95% CI |
| All PTB | 0.803 | 0.748-0.858 | 0.750 | 0.676-0.825 |
| Spontaneous | 0.806 | 0.748-0.864 | 0.837 | 0.770-0.903 |
| Provider initiated | 0.919 | 0.862-0.976 | 0.858 | 0.771-0.944 |
| <32 | 0.837 | 0.777-0.897 | 0.806 | 0.717-0.896 |
| Spontaneous | 0.840 | 0.775-0.904 | 0.868 | 0.789-0.948 |
| Provider initiated | 0.927 | 0.818-1.000 | 0.878 | 0.738-1.000 |
| 34-36 | 0.790 | 0.718-0.862 | 0.827 | 0.748-0.906 |
| Spontaneous | 0.801 | 0.723-0.880 | 0.907 | 0.843-0.971 |
| Provider initiated | 0.932 | 0.871-0.995 | 0.893 | 0.796-0.989 |
| Preeclampsia | 0.889 | 0.822-0.956 | 0.883 | 0.804-0.963 |
| <37 weeks | | | | |
| <32 Weeks | 0.953 | 0.899-1.000 | 0.879 | 0.782-0.976 |
| 32-36 Weeks | 0.938 | 0.877-0.998 | 0.950 | 0.882-1.000 |

sPTB spontaneous preterm birth,
PPROM preterm premature rupture of membranes,
AUC area under the receiver operating characteristic curve

Performance based on the use of combined maternal characteristics and serum markers exceed that based on the use of only characteristics or serum markers (AUC for all preterm birth using maternal age >34 and low-income status=0.620, 95% CI (0.553-0.687) in the training set and AUC=0.539 (95% CI 0.455-0.624) in the testing set; AUC for immune and growth markers only=0.777 (0.719-0.835) in the training set and AUC=0.743 (0.667-0.818) in the testing set. While performance varied some across PTB subgroups in the training and testing subsets, most AUCs were at or above 80%. One exception was in the training sample where the AUC for PTB 32-36 weeks was 0.790 (95% CI 0.718-0.862). The largest AUC observed was for preterm preeclampsia <32 weeks in the training sample (AUC =0.953, 95% CI 0.728-0.881 with an AUC of 0.879 (95% CI 0.782-0.976 in the testing sample) (see Table 5).

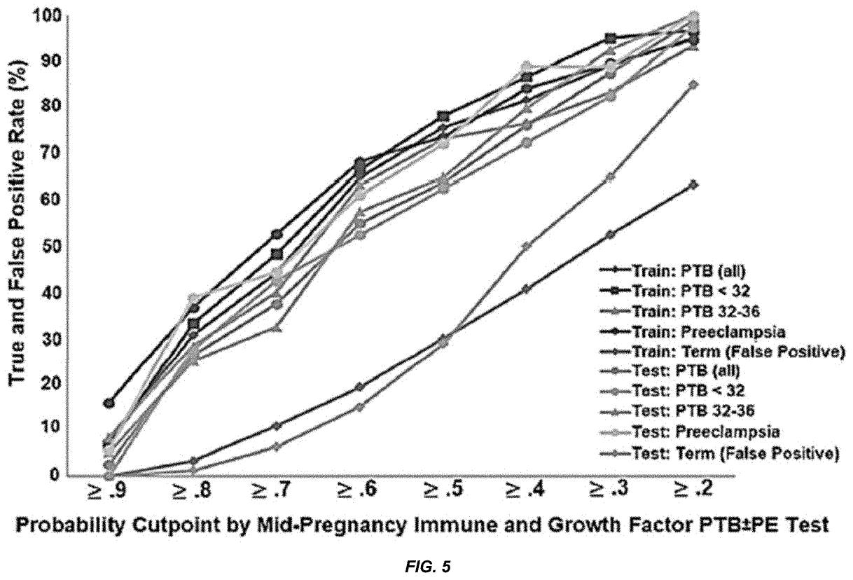

LDA-derived probabilities from the PTB±preeclampsia model yielded findings showing that the relationship between risk scores and PTB±preeclampsia overall and by subtype was consistent across the training and testing subsets with improvements in detection at each lowering of the probability cut point also associated with an increase in term false positives (see FIG. 5, see also Table 6).

TABLE 6
Frequency of preterm birth ± preeclampsia overall and by timing subgroup and
term birth by probability cut points generated by the linear discriminate function.

| | Training Sample (n = 240) | | | | | | | | | |
|---|---|---|---|---|---|---|---|---|---|---|
| | Preterm | | | | | | | | | |
| | <37 w | | <32 w | | 32-36 w | | PE | | Term | |
| | n = (%) | | n = (%) | | n = (%) | | n = (%) | | n = (%) | |
| Sample | 120 | (100.0) | 60 | (100.0) | 60 | (100.0) | 19 | (100.0) | 120 | (100.0) |
| ≥.9 | 9 | (7.5) | 4 | (6.7) | 5 | (8.3) | 3 | (15.8) | 0 | |
| ≥.8 | 37 | (30.8) | 20 | (33.3) | 17 | (28.3) | 7 | (36.8) | 4 | (3.3) |
| ≥.7 | 53 | (44.2) | 29 | (48.3) | 24 | (40.0) | 10 | (52.6) | 13 | (10.8) |
| ≥.6 | 78 | (65.0) | 40 | (66.7) | 38 | (63.3) | 13 | (68.4) | 23 | (19.2) |
| ≥.5 | 91 | (75.8) | 47 | (78.3) | 44 | (73.3) | 14 | (73.7) | 36 | (30.0) |
| ≥.4 | 98 | (81.7) | 52 | (86.7) | 46 | (76.7) | 16 | (84.2) | 49 | (40.8) |
| ≥.3 | 107 | (89.2) | 57 | (95.0) | 50 | (83.3) | 17 | (89.5) | 63 | (52.5) |
| ≥.2 | 114 | (95.0) | 58 | (96.7) | 56 | (93.3) | 18 | (94.7) | 76 | (63.3) |
| <.2 | 6 | (5.0) | 2 | (3.3) | 4 | (6.7) | 1 | (5.3) | 44 | (36.7) |

Testing Sample (n = 160)

| | Preterm | | | | | | | | | |
|---|---|---|---|---|---|---|---|---|---|---|
| | <37 w | | <32 w | | 32-36 w | | PE | | Term | |
| | n = (%) | | n = (%) | | n = (%) | | n = (%) | | n = (%) | |
| Sample | 120 | (100.0) | 60 | (100.0) | 60 | (100.0) | 18 | (100.0) | 120 | (100.0) |
| ≥.9 | 2 | (2.5) | 0 | | 2 | (5.0) | 1 | (5.6) | 0 | |
| ≥.8 | 21 | (26.3) | 11 | (27.5) | 10 | (25.0) | 7 | (38.9) | 1 | (1.3) |
| ≥.7 | 30 | (37.5) | 17 | (42.5) | 13 | (32.5) | 8 | (44.4) | 5 | (6.3) |
| ≥.6 | 44 | (55.0) | 21 | (52.5) | 23 | (57.5) | 11 | (61.1) | 12 | (15.0) |
| ≥.5 | 51 | (63.8) | 25 | (62.5) | 26 | (65.0) | 13 | (72.2) | 23 | (28.8) |
| ≥.4 | 61 | (76.3) | 29 | (72.5) | 32 | (80.0) | 16 | (88.9) | 40 | (50.0) |
| ≥.3 | 70 | (87.5) | 33 | (82.5) | 37 | (92.5) | 16 | (88.9) | 52 | (65.0) |
| ≥.2 | 79 | (98.8) | 39 | (97.5) | 40 | (100.0) | 18 | (100.0) | 68 | (85.0) |

| | | | | | | | | |
|---|---|---|---|---|---|---|---|---|
| <.2 | 1 | (1.3) | 1 | (2.5) | 0 | 0 | 12 | (15.0) |

Abbreviations: w, weeks; PE, preeclampsia.

Detection was generally better for PTBs <32 weeks and for preterm preeclampsia at each cut point than it was for PTBs from 32 to 36 weeks. For example, 30.8% of women with PTBs in the training sample and 27.5% of women with PTBs in the testing sample had probability scores≥0.8 vs. 3.3% of women with term birth in the training sample and 1.3% of term birth in the testing sample (see FIG. 5, see also Table 6). Detection at this same cut point was best in women with a PTB <32 weeks and in women with preterm preeclampsia in both samples (33.3% in the training and 27.5% in the testing samples for PTB <32 weeks and 36.8% in the training sample and 38.9% in testing sample for preterm preeclampsia) (FIG. 5, see also Table 6).

Generation of Model 1 and Derivation of Risk Indicators 1-27

Methods: Sixty-three immune- and growth-related markers were tested using a Luminex 200 instrument in banked 15-20 gestational week serum samples collected as part of routine prenatal screening by the California Genetic Disease Screening Program for 200 women with PPTB <37 weeks and 200 term controls with division into a training sample of 120 cases and 120 controls and into a testing sample of 80 cases and 80 controls. Multivariate backward stepwise logistic regression was used to identify candidate markers and linear discriminate analysis (LDA) was used to create a predictive function for PPTB. Resulting LDA probabilities were used to assess predictive capability for PPTB overall and across subtypes in the training and testing subsets using area under the curve (AUC) statistics.

Results: When combined, twenty-five immune- and growth-related markers [see footnote of Table 2] were able to identify 80.2% of women who went on to have a PPTB (AUC=0.8026, 95% CI 0.7478-0.8575) in the training sample and 73.9% in testing sample (AUC 0.7394, 0.6639-0.8149)) [see Table 5]. Performance was better in the PPTB <32 week subgroups in the training and testing samples with AUCs exceeding 80% in both (AUC=0.8368, 95% CI 0.7767 -0.8970; AUC=0.8166, 95% CI 0.7409 - 0.8922). This same algorithm identified pregnancies that developed preeclampsia with >85% accuracy across samples (AUC=0.8890, 95% CI 0.8222-0.9589; AUC 0.8794, 95% CI 0.7677-0.9911).

It will be understood that various modifications may be made without departing from the spirit and scope of this disclosure. Accordingly, other embodiments are within the scope of the following claims.

1. A method of generating a risk assessment score for preterm birth (all subtypes)±preeclampsia, for a biological sample obtained from a pregnant female subject, comprising:
measuring the level of a panel of immune and/or growth-related biomarkers from a biological sample obtained from a pregnant female subject;
assigning a risk indicator value or predictor for each of the measured immune and/or growth-related biomarkers;
inputting the obtained risk indicator values into a computer implemented predicative multivariate logistic model that is built using a training set and a testing set from a population of pregnant female subjects that comprise subjects that had preterm births and subjects that did not have preterm births; and
calculating a risk assessment score for the biological sample obtained from a pregnant female subject using the predictive model, wherein the panel of immune and/or growth-related biomarkers comprises the biomarkers for Resistin, sFASL, FGF-Basic, and SCF.

2. The method of claim 1, wherein the panel of immune and/or growth-related biomarkers further comprises biomarkers for GP130, ENA-78, NGF, PDGFBB, MIG and IL-4.

3. The method of claim 2, wherein the panel of immune and/or growth-related biomarkers further comprises biomarkers for IL-4R, IL-5, IL-13, IL-17, RAGE, VEGFR3, and RANTES.

4. The method of claim 3, wherein the panel of immune and/or growth-related biomarkers further comprises biomarkers for PAI1, G-CSF, IL-1 R2, IL-17F, IFNB, M-CSF, Eotaxin, and MIP1B.

5. The method of claim 1, wherein the panel of immune and/or growth-related biomarkers consists essentially of Resistin, sFASL, FGF-Basic, SCF, GP130, ENA-78, NGF, PDGFBB, MIG, IL-4, IL-4R, IL-5, IL-13, IL-17, RAGE, VEGFR3, RANTES, PAI1, G-CSF, IL-1R2, IL-17F, IFNB, M-CSF, Eotaxin, and MIP1B.

6. The method of claim 1, wherein the panel of immune and/or growth-related biomarkers consists of Resistin, sFASL, FGF-Basic, SCF, GP130, ENA-78, NGF, PDGFBB, MIG, IL-4, IL-4R, IL-5, IL-13, IL-17, RAGE, VEGFR3, RANTES, PAI1, G-CSF, IL-1R2, IL-17F, IFNB, M-CSF, Eotaxin, and MIP1B.

7. The method of claim 1, wherein the biological sample is a serum sample.

8. The method of claim 1, wherein the biological sample is a sample obtained from a pregnant female subject that has less than 32 weeks of gestation.

9. The method of claim 1, wherein the biological sample is a sample obtained from a pregnant female subject that 15 to 20 weeks of gestation.

10. The method of claim 1, wherein the panel of biomarkers are measured using a quantitative multiplex assay.

11. The method of claim 10, wherein the quantitative multiplex assay is a quantitative bead-based multiplex immunoassay.

12. The method of claim 1, wherein the predicative multivariate logistic model is a linear discriminant analysis model.

13. The method of claim 12, wherein the linear discriminant analysis model uses the coefficients for the biomarkers presented in Table 1.

14. The method of claim 1, wherein the predictive multivariate logistic model uses the coefficients for the biomarkers presented in Table 1.

15. The method of claim 1, where the method further comprises:
assessing the pregnant female subject for any secondary risk factors, including maternal characteristics, medical history, past pregnancy history, obstetrical history, income status, alcohol, tobacco or drug use, diabetes, hypertension, and interpregnancy interval;
assigning a risk indicator value for each secondary risk factors;
inputting the obtained risk indicator values for the secondary risk factors along with the obtained risk indicator values for the biomarkers into the computer implemented predicative multivariate logistic model; and
calculating a risk assessment score for the biological sample obtained from a pregnant female subject using the predictive model.

16. The method of claim 15, wherein the method uses risk indicator values or predictors for the pregnant female subject being >34 years of age, and/or for the pregnant female subject having a low-income status.

17. A method for prophylactically treating a pregnant female subject for preterm birth, comprising:
determining a risk assessment score from a biological sample obtained from the pregnant female subject using the method of claim 4;
administering a treatment to the pregnant female subject if the risk assessment score for the subject sample indicates that the subject has a high probability for preterm birth,
wherein the treatment is selected from progesterone, cervical pessary, cervical cerclage, tocolytic administration, and antibiotic therapy.

18. A kit for assessing preterm birth and preeclampsia risk biomarkers in a sample, wherein the kit comprises a detecting agent(s) for each biomarker in a panel of biomarkers consisting essentially of Resistin, sFASL, FGF-Basic, SCF, GP130, ENA-78, NGF, PDGFBB, MIG, IL-4, IL-4R, IL-5, IL-13, IL-17, RAGE, VEGFR3, RANTES, PAI1, G-CSF, IL-1R2, IL-17F, IFNB, M-CSF, Eotaxin, and MIP1B.

19. The kit of claim 18, wherein the detecting agents are antibodies.

20. The kit of claim 19, wherein the kit is an ELISA or antibody microarray.
